(19) United States
(12) Patent Application Publication
(10) Pub. No.: US 2004/0167334 A1
(43) Pub. Date: Aug. 26, 2004

(54) Heterocyclic acylsulfimides, processes for their preparation, compositions comprising them and their use as pesticides

(76) Inventors: Yuriy Grigorievich Shermolovich, Kiev (UA); Pavel Petrovich Kornuta, Kiev (UA); Nataliya Olezandrivna Komuta, Kiev (UA); Uwe Doller, Rodgau (DE); Oswald Ort, Glashutten (DE); Wolfgang Schaper, Diedorf (DE); Daniela Jans, Bad Homburg v. d. H. (DE); Ulrich Sanft, Eppstein/Ts (DE); Maria-Theresia Thonessen, Heidesheim (DE); Marion Beckmann, Wiesbaden (DE); Jutta Maria Waibel, Frankfurt (DE); Sergiy Pazenok, Kelkheim (DE)

Correspondence Address: FROMMER LAWRENCE & HAUG, 745 FIFTH AVENUE- 10TH FL., NEW YORK, NY, 10151, US

(21) Appl. No.: 10/773,471

(22) Filed: Feb. 5, 2004

Related U.S. Application Data
(63) Continuation of application No. 09/812,309, filed on Mar. 20, 2001, now abandoned.

(30) Foreign Application Priority Data
Mar. 22, 2000 (DE) 10014006.8
Nov. 21, 2000 (DE) 10057911.6

(51) Int. Cl. C07D 239/02

(52) U.S. Cl. 544/316; 544/335

(57) ABSTRACT

Heterocyclic acylsulfimides, processes for their preparation, compositions comprising them and their use as pesticides

Acylsulfimides of the formula (I)

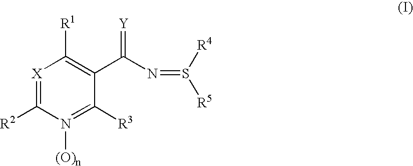

where the symbols and indices are as defined in the description, are suitable for controlling animal pests.

The invention relates to heterocyclic acylsulfimides, to processes for their preparation, to compositions comprising them and to their use for controlling animal pests, in particular arthropods, such as insects and acarids, and helminths.

Owing to the enormous damage caused by insects, for example by feeding on useful plants, stored food, wood and textiles, or else by transferring diseases to man, domestic animals and useful plants, the use of insecticides or repellents is still indispensable. Insecticides are an important component of integrated pest control, and their contribution is decisive with respect to harvest yield and yield continuity all over the world.

EP-A 0 580 374 discloses trifluoromethylpyridine amides for use as pesticides.

However, since the ecological and economical demands made on modern insecticides are increasing permanently, for example with respect to toxicity, selectivity, application rate, formation of residues and favorable manufacture and there can furthermore be problems, for example with resistance, there is a constant need to develop novel insecticides which, at least in some areas, have advantages over those of the prior art.

It has been found that compounds of the formula (I), if appropriate also as salts, have a good activity spectrum against animal pests and at the same time good plant tolerance and favorable toxicological properties with respect to mammals and aquatic animals.

Sulfimides of the unsubstituted nicotinamide are disclosed in Fiziol Biokhim Kul't Rast (1999) 31 (4) 303-307, as safeners for herbicides. The suitability of the 4-haloalkyl substituted compounds as insecticides cannot be deducted from this.

Accordingly, the invention provides acylsulfimides and -sulfoxyimides of the formula (I) and salts thereof,

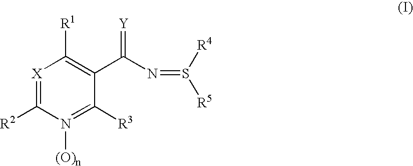

where the symbols and indices are as defined below:

x is CH or N;

Y is O or S;

n is 0 or 1;

m is 0 or 1;

R¹ is C₁-C₆-haloalkyl;

R², R³ are identical or different and are H, halogen or a branched or unbranched (C₁-C₆)-alkyl group, where one or two CH₂ groups may be replaced by —O— or —S— or —N(C₁-C₆)-alkyl, with the proviso that heteroatoms may not be adjacent to one another;

R⁴, R⁵ are identical or different and are R⁶, —C(LW)R⁷, —C(═NOR⁷)R⁷, —C(═NNR⁷₂)R⁷, —C(═W)OR⁷, —C(═W)NR⁷₂, —OC(═W)R⁷, —OC(═W)OR⁷, —NR⁷C(═W)R⁷, —N [C(═W)R⁷]₂, —NR⁷C(═W)OR⁷, —C(═W)NR⁷—NR⁷₂, —C(═W)NR⁷—NR⁷[C(═W)R⁷], —NR⁷—C(═W)NR⁷₂, —NR⁷—NR⁷C(═W)R⁷, —NR⁷—N[C(═W)R⁷]₂, —N[(C═W)R⁷]—NR⁷₂, —NR⁷—NR⁷[(C═W)WR⁷], —NR⁷[(C═W)NR⁷]₂, —NR⁷(C═NR⁷)R⁷, —NR⁷(C═NR⁷)NR⁷₂, —O—NR⁷₂, —O—NR⁷(C═W)R⁷, —SO₂NR⁷₂, —NR⁷SO₂R⁷, —SO₂OR⁷, —OSO₂R⁷, —OR⁷, —NR⁷₂, —SR⁷, —SiR⁷₃, —PR⁷₂, —P(═W)R⁷, —SOR⁷, —SO₂R⁷—PW₂R⁷₂, —PW₃R⁷₂;

or

R⁴ and R⁵ together with the sulfur to which they are attached form a three- to eight-membered saturated or unsaturated, preferably carbocyclic ring system which is optionally mono- or polysubstituted, preferably by radicals R⁸, and which optionally contains 1 to 4 further heteroatoms, where two or more of the substituents optionally form one or more further ring systems;

W is O or S;

R⁸ are identical or different and are (C₁-C₂₀)-alkyl, (C₂-C₂₀)-alkenyl, (C₂-C₂₀)alkynyl, (C₃-C₈)-cycloalkyl, (C₄-C₈)cycloalkenyl, (C₈-C₁₀)-cycloalkynyl, aryl or heterocyclyl, where the radicals mentioned may optionally be mono- or polysubstituted, preferably by radicals R⁸;

R⁷ is identical or different and is H or R⁶.

The symbols and indices in the formula (I) are preferably as defined below:

X is preferably CH.

Y is preferably 0.

m is preferably 0.

n is preferably 0.

R¹ is preferably (C₁-C₈)-alkyl which is mono- or polysubstituted by F and/or —Cl, particularly preferably CF₃, CHF₂ or CF₂Cl, very particularly preferably CF₃.

R², —R³ are preferably H, halogen, N(C₁-C₆)₂-alkyl, particularly preferably H.

R⁴, R⁵ are preferably OR⁷, NR⁷₂ or R⁷.

Particular preference is given to those compounds of the formula (I), in which the symbols and indices are as defined below:

X is preferably CH.

Y is preferably 0.

m is preferably 0.

n is preferably 0.

R¹ is preferably CF₃.

R², R³ are preferably H.

R⁴, R⁵ are preferably R⁶.

Preferred substituents on the radicals R⁴, R⁵ are groups R⁸, which are as defined below:

R⁸ are identical or different and are R⁹, or two radicals R⁸ together with the atoms to which they are attached form a three- to eight-membered saturated or unsaturated ring system, optionally substituted by one or more radicals R⁹, which optionally also contains further heteroatoms, preferably from the group O, N, S, SO and SO₂;

R⁹ are identical or different and are R¹⁰, R¹¹, —C(W)R¹⁰, —C(═NOR¹⁰)R¹⁰, —C(═N NR¹⁰₂)R¹⁰, —C(═W)OR¹⁰, —C(═W)NR¹⁰₂, —OC(═W)R¹⁰, —OC(═W)OR¹⁰, —NR¹⁰C(═W)R¹⁰, —N[C(═W)R¹⁰]₂, —NR¹⁰C(═W)OR¹⁰, —C(═W)NR¹⁰—NR¹⁰₂, —C(═W)NR¹⁰—NR¹⁰[C(═W)R¹⁰], —NR¹⁰—C(═W)NR¹⁰₂, —NR¹⁰—NR¹⁰C(═W)R¹⁰, —NR¹⁰—N[C(═W)R¹¹]₂, —N[(C═W)R¹⁹—NR¹⁰]₂, —NR¹⁰—N[(C═W)WR¹⁰], —NR¹⁰ [(C═W)NR¹⁰], —NR¹⁰(C═NR¹⁰)R¹⁰, —NR¹⁰(C═NR¹⁰)NR¹⁰₂, —O—NR¹⁰₂, —O—NR¹⁰(C═W)R¹⁰, —SO₂NR¹⁰₂, —NR¹⁰SO₂R¹⁰, —SO₂OR¹⁰, —OSO₂R¹⁰, —OR¹⁰, —NR¹⁰₂, —SR¹⁰, —SiR¹⁰ ₃, —PR¹⁰₂, —P(═W)R¹⁰², —SOR¹⁰, —SO₂R¹⁰, —PW₂R¹⁰₂, —PW₃R¹⁰₂; or two radicals R⁹ together form (═W), (═NR¹⁰), (═CR₂¹⁰), (═CHR¹⁰) or (═CH₂);

R¹⁰ are identical or different and are (C₁-C₆)-alkyl, (C₂-C₆)-alkenyl, (C₂-C₆)-alkynyl, (C₃-C₈)cycloalkyl, (C₄-C₈)-cycloalkenyl, (C₃-C₈)-cycloalkyl-(C₁-C₄)-alkyl, (C₄-C₈)-cycloalkenyl-(C₁-C₄)-alkyl, (C₃-C₈)-cycloalkyl-(C₂-C₄)-alkenyl, (C₄-C₈)-cycloalkenyl-(C₂-C₄)-alkenyl, (C₁-C₆)-alkyl-(C₃-C₈)-cycloalkyl, (C₂-C₆)-alkenyl-(C₃-C₈)-cycloalkyl, (C₂-C₆)-alkynyl-(C₃-C₈)-cycloalkyl, (C₁-C₆)-alkyl-(C₄-C₈)-cycloalkenyl, (C₂-C₆)-alkenyl-(C₄-C₈)-cycloalkenyl, aryl, heterocyclyl;

where the radicals mentioned are optionally substituted by one or more radicals R¹¹;

R¹¹ are identical or different and are halogen, cyano, nitro, hydroxyl, thio, amino, formyl, (C₁-C₆)-alkanoyl, (C₁-C₆)-alkoxy, (C₃-C₆)-alkenyloxy, (C₃-C₆)-alkynyloxy, (C₁-C₆)-haloalkyloxy, (C₃-C₆)-haloalkenyloxy, (C₃-C₆)-haloalkynyloxy, (C₃-C₈)-cycloalkoxy, (C₄-C₈)-cycloalkenyloxy, (C₃-C₈) halocycloalkoxy, (C₄-C₈)-halocycloalkenyloxy, (C₃-C₈)-cycloalkyl-(C₁-C₄)-alkoxy,

(C₄-C₈)-cycloalkenyl-(C₁-C₄)-alkoxy, (C₃-C₈)-cycloalkyl-(C₂-C₄) alkenyloxy, (C₄-C₈)-cycloalkenyl-(C₁-C₄)-alkenyloxy, (C₁-C₆)-alkyl-(C₃-C₈)-cycloalkoxy, (C₂-C₆)-alkenyl-(C₃-C₈)-cycloalkoxy, (C₂-C₆)-alkynyl-(C₃-C₈)-cycloalkoxy, (C₁-C₆-alkyl-(C₄-C₈)-cycloalkenyloxy, (C₂-C₆)-alkenyl-(C₄-C₈)-cycloalkenyloxy, (C₁-C₄)-alkoxy-(C₁-C₆)-alkoxy, (C₁-C₄)-alkoxy-(C₃-C₆)-alkenyloxy, carbamoyl, (C₁-C₆)-mono- or dialkylcarbamoyl, (C₁-C₆)-mono- or dihaloalkylcarbamoyl, (C₃-C₈)-mono- or dicycloalkylcarbamoyl, (C₁-C₆)-alkoxycarbonyl, (C₃-C₈)-cycloalkoxycarbonyl, (C₁-C₆)-alkanoyloxy, (C₃-C₈)-cycloalkanoyloxy, (C₁-C₆)-haloalkoxycarbonyl, (C₁-C₆)-haloalkanoyloxy, (C₁-C₆)-alkanamido, (C₁-C₆)-haloalkanamido, (C₂-C₆)-alkenamido, (C₃-C₈)-cycloalkanamido, (C₃-C₈)-cycloalkyl-(C₁-C₄-alkanamido, (C₁-C₆)-alkylthio, (C₃-C₆)-alkenylthio, (C₃-C₆)-alkynylthio, (C₁-C₆) haloalkylthio-(C₃-C₆)-haloalkenylthio, (C₃-C₆)haloalkynylthio, (C₃-C₈)-cycloalkylthio, (C₄-C₈)cycloalkenylthio, (C₃-C₈)-halocycloalkylthio, (C₄-C₈)-halocycloalkenylthio, (C₃-C₈)-cycloalkyl-(C₁-C₄)alkylthio, (C₄-C₈)-cycloalkenyl-(C₁-C₄)-alkylthio, (C₃-C₈)-cycloalkyl-(C₃-C₄) alkenylthio, (C₄-C₈)cycloalkenyl-(C₃-C₄)alkenylthio, (C₁-C₆)-alkyl-(C₃-C₈)-cycloalkylthio, (C₂-C₆)-alkenyl-(C₃-C₈)-cycloalkylthio, (C₂-C₆)-alkynyl-(C₃-C₈)-cycloalkylthio, (C₁-C₆)-alkyl-(C₄-C₈)-cycloalkenylthio, (C₂-C₆)-alkenyl-(C₄-C₈)-cycloalkenylthio, (C₁-C₆)-alkylsulfinyl, (C₃-C₆)-alkenylsulfinyl, (C₃-C₆)-alkynylsulfinyl, (C₁-C₆)haloalkylsulfinyl, (C₃-C₆) haloalkenylsulfinyl, (C₃-C₆)-haloalkynylsulfinyl, (C₃-C₈)-cycloalkylsulfinyl, (C₄-C₈)-cycloalkenylsulfinyl, (C₃-C₈)-halocycloalkylsulfinyl, (C₄-C₈)-halocycloalkenylsulfinyl, (C₃-C₈)-cycloalkyl-(C₁-C₄-alkylsulfinyl, (C₄-C₈)-cycloalkenyl-(C₄-C₈)-alkylsulfinyl, (C₃-C₈)-cycloalkyl-(C₃-C₄) alkenylsulfinyl, (C₄-C₈)-cycloalkenyl-(C₃-C₄)-alkenylsulfinyl, (C₁-C₆-alkyl-(C₃-C₈)-cycloalkylsulfinyl, (C₂-C₆)-alkenyl-(C₃-C₈)-alkylsulfinyl, (C₂-C₆)-alkynyl-(C₃-C₆)-cycloalkylsulfinyl, (C₁-C₆)-alkyl-(C₄-C₈)— cycloalkenylsulfinyl, (C₂-C₆)-alkenyl-(C₄-C₈)-cycloalkenylsulfinyl, (C₁-C₆)-alkylsulfonyl, (C₃-C₆)-alkenylsulfonyl, (C₃-C₆)-alkynylsulfonyl, (C₁-C₆)-haloalkylsulfonyl, (C₃-C₆)-haloalkenylsulfonyl, (C₃-C₆)-haloalkynylsulfonyl, (C₃-C₈)-cycloalkylsulfonyl, (C₄-C₈) cycloalkenylsulfonyl, (C₃-C₈)-halocycloalkylsulfonyl, (C₄-C₈)-halocycloalkenylsulfonyl, (C₃-C₆)-cycloalkyl-(C₁-C₄)-alkylsulfonyl, (C₄-C₈)-cycloalkenyl-(C₁-C₄)-alkylsulfonyl, (C₃-C₈)-cycloalkyl-(C₃-C₄)-alkenylsulfonyl, (C₄-C₈)cycloalkenyllkenyisulfonyl, (C₁-C₆) alkyl-(C₃-C₈)-cycloalkylsulfonyl, (C₂-C₆)-alkenyl-(C₃-C₈)-cycloalkylsulfonyl, (C₂-C₆)-alkynyl-(C₃-C₈)-cycloalkylsulfonyl, (C₁-C₆) alkyl-(C₄-C₈)-cycloalkenylsulfonyl, (C₂-C₆)-alkenyl-(C₄-C₈, cycloalkenylsulfonyl, (C₁-C₆)-dialkylamino, (C₁-C₆)-alkylamino, (C₃-C₄)-alkenylamino, (C₃-C₆)-alkynylamino, (C₁-C₆)-haloalkylamino, (C₃-C₆)-haloalkenylamino, (C₃-C₆)-haloalkynylamino, (C₃-C₈)cycloalkylamino, (C₄-C₈)-cycloalkenylamino, (C₃-C₈)-halocycloalkylamino, (C₄-C₈)-halocycloalkenylamino, (C₃-C₈)-cycloalkyl-(C₁-C₄)-alkylamino, (C₄-C₈ cycloalkenyl-(C₁-C₄)-alkylamino, (C₃-C₈)cycloalkyl-(C₃-C₄) alkenylamino, (C₄-C₈)-cycloalkenyl-(C₃-C₄)-alkenylamino, (C₁-C₆)-alkyl-(C₃-C₈)-cycloalkylamino, (C₂-C₆)-alkenyl-(C₃-C₈)-cycloalkylamino, (C₂-C₆)-alkynyl-(C₃-C₈)-cycloalkylamino, (C₁-C₆)-alkyl-(C₄-C₈)-cycloalkenylamino, (C₂-C₆)-alkenyl-(C₄-C₈)-cycloalkenylamino, (C₁-C₆) trialkylsilyl, aryl, aryloxy, arylthio, arylamino, aryl-(C₁-C₄)-alkoxy, aryl-(C₃-C₄)-alkenyloxy, aryl-(C₁-C₄)-alkylthio, aryl-(C₂-C₄)-alkenylthio, aryl-(C₁-C₄)-alkylamino, aryl-(C₃-C₄)-alkenylamino, aryl-(C₁-C₆)-dialkylsilyl, diaryl-(C₁-C₆)-alkylsilyl, triarylsilyl and 5- or 6-membered heterocyclyl, the cyclic moiety of the fourteen last-mentioned radicals being optionally substituted by one or more radicals selected from the group consisting of halogen, cyano, nitro, amino, hydroxyl, thio, (C₁-C₄)alkyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylamino, (C₁-C₄) haloalkylamino, formyl and (C₁-C₄)-alkanoyl.

R¹¹ are preferably identical or different and are halogen, cyano, (C₁-C₆)— alkanoyl, (C₁-C₆)-alkoxy, (C₁-C₆)-haloalkyloxy, (C₃-C₈)-cycloalkoxy, —(C₃-C₈)-cycloalkyl-(C₁-C₄)-alkoxy, (C₁-C₆)-mono- or dialkylcarbamoyl, (C₁-C₆)-alkanoyloxy, (C₁-C₆)-haloalkoxycarbonyl, (C₁-C₆)-alkylthio, (C₁-C₆)— haloalkylthio, (C₃-C₈)-cycloalkylthio, (C₁-C₆)-alkylsulfinyl, (C₁-C₆)— haloalkylsulfinyl, (C₃-C₈)-cycloalkylsulfinyl, (C₁-C₆)-alkylsulfonyl, (C₁-C₆)— haloalkylsulfonyl, (C₃-C₈)-cycloalkylsulfonyl, (C₁-C₆)-dialkylamino, (C₁-C₆)-alkylamino, (C₃-C₈)-cycloalkylamino, (C₁-C₆)-trialkylsilyl, aryl, aryloxy, arylthio, arylamino, (aryl)-(C₁-C₄)-alkyl, aryl-(C₁-C₄)-alkoxy, the cyclic moiety of the six last-mentioned radicals being optionally substituted by one or more radicals selected from the group consisting of halogen, nitro, (C₁-C₄)-alkyl, (C₁-C₄)-haloalkyl, (C₁-C₄)-alkoxy, (C₁-C₄)-haloalkoxy.

Among the radicals R⁴ and R⁵, particular preference is given to those for which the unit R⁴R⁵ is represented by the following structures:

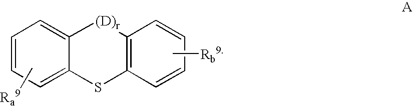

wherein the symbols and indices have the following meanings:

r is 0, 1;

D is a direct bond, (C₁-C₄ alkylene, branched or unbranched, O, S(O)_{0,1,2}, or NR¹¹;

R⁹ is a substituent covered by the description;

R¹¹ is H, (C₁-C₄)-alkyl, branched or unbranched, (C₁-C₄)-alkanoyl, (C₁-C₄)— alkoxycarbonyl, (C₁-C₄)-alkyl- or -dialkylaminocarbonyl or (C₁-C₄)-alkylsulfonyl.

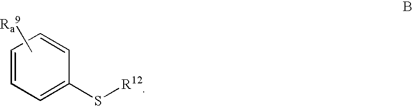

wherein the symbols and indices have the following meanings:

R¹² is (C₁-C₈)-alkyl, optionally substituted by an optionally substituted phenyl radical or (C₃-C₆)-cycloalkyl radical, (C₃-C₆)-cycloalkyl, optionally substituted by or condensed with an optionally substituted phenyl radical;

R⁹ are substituents covered by the description;

a is 0, 1, 2, 3, 4, or 5, preferably 0, 1 or 2.

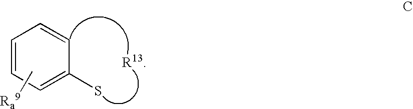

wherein the symbols and indices have the following meanings:

R⁹ is a substituent covered by the description;

a is 0, 1, 2, 3 or 4, preferably 0, 1 or 2;

R¹³ is a straight chain or branched (C₂-C₈)-alkanediyl group, optionally substituted by one or two or condensed with an optionally substituted phenyl radical.

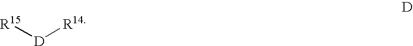

wherein the symbols and indices have the following meanings:

R¹⁴, R¹⁵ are identical or different and are in each case (C₁-C₈)-alkyl, optionally substituted by or condensed with an optionally substituted phenyl radical or (C₃-C₈)-cycloalkyl radical, (C₃-C₆)-cycloalkyl, optionally substituted by or condensed with an optionally substituted phenyl radical.

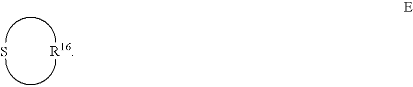

wherein the symbol has the following meaning:

R¹⁶ is a straight chain or branched (C₂-C₈)-alkanediyl group, optionally substituted by one or two or condensed with an optionally substituted phenyl radical.

The term “halogen” embraces fluorine, chlorine, bromine and iodine. The expression “(C₁-C₄)-alkyl” is to be understood as meaning an unbranched or branched hydrocarbon radical having 1, 2, 3 or 4 carbon atoms, such as, for example, the methyl, ethyl, propyl, isopropyl, 1-butyl, 2-butyl, 2-methylpropyl or tert-butyl radical. Correspondingly, alkyl radicals having a larger range of carbon atoms are to be understood as meaning an unbranched or branched saturated hydrocarbon radical which contains a number of carbon atoms which corresponds to this stated range.

Accordingly, the expression “(C₁-C₆)-alkyl” embraces the abovementioned alkyl radicals, and also, for example, the pentyl, 2-methylbutyl, 1,1-dimethylpropyl or hexyl radical. The expression “(C₁-C₁₀)-alkyl” is to be understood as meaning the abovementioned alkyl radicals, and also, for example, the nonyl, 1-decyl or 2-decyl radical.

“(C₁-C₄)-Haloalkyl” is to be understood as meaning an alkyl group mentioned under the expression “(C₁-C₄)-alkyl” in which one or more hydrogen atoms are replaced by the same number of identical or different halogen atoms, preferably by chlorine or fluorine, such as the trifluoromethyl, the 1-fluoroethyl, the 2,2,2-trifluoroethyl, the chloromethyl, fluoromethyl, the difluoromethyl and the 1,1,2,2-tetrafluoroethyl group.

“(C₁-C₄)-Alkoxy” is to be understood as meaning an alkoxy group whose hydrocarbon radical has the meaning given under the expression “(C₁-C₄)-alkyl”. Alkoxy groups having a larger range of carbon atoms are to be understood accordingly.

The terms “alkenyl” and “alkynyl” with a range of carbon atoms stated as prefix denote a straight-chain or branched hydrocarbon radical having a number of carbon atoms which corresponds to this stated range and which contains at least one multiple bond which can be located in any position of the respective unsaturated radical. “(C₂-C₄)-Alkenyl” accordingly denotes, for example, the vinyl, allyl, 2-methyl-2-propenyl or 2-butenyl group; “(C₂-C₆)-Alkenyl” denotes the above-mentioned radicals and also, for example, the pentenyl, 2-methylpentenyl or the hexenyl group. “(C₂-C₄)-Alkynyl” denotes, for example, the ethynyl, propargyl, 2-methyl-2-propynyl or 2-butynyl group. “(C₂-C₆)-Alkynyl” is to be understood as meaning the abovementioned radicals and also, for example, the 2-pentynyl or the 2-hexynyl group, and “(C₂-C₁₀)-alkynyl” is to be understood as meaning the above-mentioned radicals and also, for example, the 2-octynyl or the 2-decynyl group.

“(C₃-C₈)-Cycloalkyl” denotes monocyclic alkyl radicals, such as the cyclopropyl, cyclobutyl, cyclopentyl, cyclohexyl, cycloheptyl or cyclooctyl radical, and denotes bicyclic alkyl radicals, such as the norbornyl radical.

The expression “(C₃-C₈)-cycloalkyl-(C₁-C₄)-alkyl” is to be understood as meaning, for example the cyclopropylmethyl, cyclopentylmethyl, cyclohexylmethyl, cyclohexylethyl and cyclohexylbutyl radical, and the expression “(C₁-C₆)-alkyl-(C₃-C₈)-cycloalkyl” is to be understood as meaning, for example, the 1-methylcyclopropyl, 1-methylcyclopentyl, 1-methylcyclohexyl, 3-hexylcyclobutyl and the 4-tert-butylcyclohexyl radical.

“(C₁-C₄)-Alkoxy-(C₁-C₆)-alkyloxy” denotes an alkoxy group as defined above which is substituted by a further alkoxy group, such as, for example, 1-ethoxyethoxy.

“(C₃-C₈)-Cycloalkoxy” or “(C₃-C₈)-cycloalkylthio” is to be understood as meaning one of the abovementioned (C₃-C₈)-cycloalkyl radicals which is attached via an oxygen or sulfur atom.

“(C₃-C₈)-Cycloalkyl-(C₁-C₆)-alkoxy” denotes, for example, the cyclopropylmethoxy, cyclobutylmethoxy, cyclopentylmethoxy, cyclohexylmethoxy, cyclohexylethoxy or the cyclohexylbutoxy group.

The expression “(C₁-C₄)-alkyl-(C₃-C₈)-cycloalkoxy” denotes, for example, the methylcyclopropyloxy, methylcyclobutyloxy or the butylcyclohexyloxy group.

“(C₁-C₆)-Alkylthio” denotes an alkylthio group whose hydrocarbon radical has the meaning given under the expression “(C₁-C₆)-alkyl”.

Similarly, “(C₁-C₆)-alkylsulfinyl” denotes, for example, the methyl, ethyl, propyl, isopropyl, butyl, isobutyl, sec-butyl- or tert-butylsulfinyl group and

“(C₁-C₆)alkylsulfonyl” denotes, for example, the methyl, ethyl, propyl, isopropyl, butyl, isobutyl, sec-butyl- or tert-butylsulfonyl group.

“(C₁-C₆)-Alkylamino” denotes a nitrogen atom which is substituted by one or two identical or different alkyl radicals of the above definition.

The expression “(C₁-C₆)-mono- or -dialkylcarbamoyl” denotes a carbamoyl group having one or two hydrocarbon radicals which have the meaning given under the expression “(C₁-C₆)-alkyl” and which, in the case of two hydrocarbon radicals, can be identical or different.

Similarly, “(C₁-C₆)-dihaloalkylcarbamoyl” denotes a carbamoyl group which carries two (C₁-C₈)haloalkyl radicals according to the above definition or one (C₁-C₆, haloalkyl radical and one (C₁-C₆)-alkyl radical according to the above definition.

“(C₁-C₆)-Alkanoyl” denotes, for example, the formyl, acetyl, propionyl, butyryl or 2-methylbutyryl group.

The expression “aryl” is to be understood as meaning a carbocyclic, i.e. constructed of carbon atoms, aromatic radical having preferably 6 to 14, in particular 6 to 12, carbon atoms, such as, for example, phenyl, naphthyl or biphenylyl, preferably phenyl. “Aroyl” accordingly denotes an aryl radical as defined above which is attached via a carbonyl group, such as, for example, the benzoyl group.

The expression “heterocyclyl” preferably denotes a cyclic radical which can be completely saturated, partially unsaturated or completely unsaturated and which can be interrupted by at least one or more identical or different atoms selected from the group consisting of nitrogen, sulfur and oxygen, where, however, two oxygen atoms may not be directly adjacent and at least one carbon atom has to be present in the ring, such as, for example, a thiophene, furan, pyrrole, thiazole, oxazole, imidazole, isothiazoe, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine, 4H-quinolizine, piperidine, pyrrolidine, oxazoline, tetrahydrofuran, tetrahydropyran, isoxazolidine or thiazolidine radical. Accordingly, the expression “heteroaromatic” embraces, from among the meanings mentioned above under “heterocyclyl”, in each case the completely unsaturated aromatic heterocyclic compounds.

Heterocyclyl particularly preferably denotes a saturated, partially saturated or aromatic ring system having 3 to 6 ring members and 1 to 4 heteroatoms selected from the group consisting of O, S and N, where at least one carbon atom has to be present in the ring.

Very particularly preferably, heterocyclyl denotes a pyridine, pyrimidine, (1,2,4)-oxadiazole, (1,3,4)-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydrofuran, tetrahydropyran, morpholine, piperidine, piperazine, pyrroline, pyrrolidine, oxazolidine, thiazolidine, oxirane and oxetane radical.

“Aryl-(C₁-C₄)-alkoxy” denotes an aryl radical which is attached via a (C₁-C₄)-alkoxy group, for example the benzyloxy, phenylethoxy, phenylbutoxy or naphthylmethoxy radical.

“Arylthio” denotes an aryl radical which is attached via a sulfur atom, for example the phenylthio or the 1- or 2-naphthylthio radical. Similarly, “aryloxy” denotes, for example, the phenoxy or 1- or 2-naphthyloxy radical.

“Aryl-(C₁-C₄)-alkylthio” denotes an aryl radical which is attached via an alkylthio radical, for example the benzylthio, naphthylmethylthio or the phenylethylthio radical.

The expression “(C₁-C₆)-trialkylsilyl” denotes a silicon atom which carries three identical or different alkyl radicals according to the above definition. Similarly, “aryl-(C₁-C₆)-dialkylsilyl” denotes a silicon atom which carries one aryl radical and two identical or different alkyl radicals according to the above definition, “diaryl-(C₁-C₆) alkylsilyl” denotes a silicon atom which carries one alkyl radical and two identical or different aryl radicals according to the above definition and “triarylsilyl” denotes a silicon atom which carries three identical or different aryl radicals according to the above definition.

Preferred substituents for the various aliphatic, aromatic and heterocyclic ring systems include halogen, nitro, cyano, di-(C₁-C₄)-alkylamino, (C₁-C₄)-alkyl, (C₁-C₄) trialkylsilyl, (C₁-C₄)-alkoxy, (C₁-C₄)-alkoxy-(C₁-C₄)-alkyl, (C₁-C₂)-alkoxy-[CH₂CH₂]_{1,2}-ethoxy, (C₁-C₄)-alkylthio, (C₁-C₄)-alkylsulfinyl, (C₁-C₄)-alkylsulfonyl, phenyl, benzyl, phenoxy, phenylthio, halophenoxy, (C₁-C₄)-alkylphenoxy, (C₁-C₄)-alkoxyphenoxy, (C₁-C₄)-alkylthiophenoxy, phenylthio, heterocyclyl, heterocyclylthio, heterocyclyloxy, haloheterocyclyloxy, alkylheterocyclyloxy or alkoxyheterocyclyloxy, where in the alkyl radicals and the radicals derived therefrom one or more—and in the case of fluorine up to the maximum number of—hydrogen atoms can be replaced by halogen, preferably chlorine or fluorine.

Particularly preferred substituents, especially in the case of cyclic systems, include halogen, nitro, cyano, amino, hydroxy, thio, (C₁-C₄)-alkyl, (C₁-C₄)-haloalkyl, (C₃-C₈)— cycloalkyl, (C₁-C₄)-alkoxy, (C₁-C₄)-haloalkoxy, (C₁-C₄)-alkylthio, (C₁-C₄)-haloalkylthio, (C₁-C₄)-alkylamino, (C₁-C₄)-haloalkylamino, formyl and (C₁-C₄)-alkanoyl.

Depending on the nature of the substituents defined above, the compounds of the formula (I) have acidic or basic properties and are capable of forming salts. If, for example, the compounds of the formula (I) carry groups such as hydroxyl, carboxyl or other groups which induce acidic properties, these compounds can be reacted with bases to form salts. Suitable bases are, for example, hydroxides, carbonates and bicarbonates of the alkali metals and alkaline earth metals, in particular those of sodium, potassium, magnesium and calcium, furthermore ammonia, primary, secondary and tertiary amines with (C₁-C₄)-alkyl radicals and mono-, di- and trialkanolamines of (C₁-C₄)-alkanols. If, for example, the compounds of the formula (I) carry groups such as amino, alkylamino or other groups which induce basic properties, these compounds can be reacted with acids to form salts. Suitable acids are, for example, mineral acids, such as hydrochloric, sulfuric and phosphoric acid, organic acids, such as acetic acid or oxalic acid, and acidic salts, such as NaHSO₄ and KHSO₄. The salts obtainable in this manner likewise have insecticidal, acaricidal and miticidal properties.

The compounds of the formula (I) can have an asymmetrically substituted sulfur atom and/or one or more asymmetrically substituted carbon atoms or stereoisomers on double bonds. Therefore, it is possible for enantiomers or diastereomers to be present. The invention embraces both the pure isomers and their mixtures. The mixtures of diastereomers can be separated into the isomers by customary methods, for example by selective crystallization from suitable solvents or by chromatography. Racemates can be separated into the enantiomers by customary methods.

The present invention also relates to processes for preparing compounds of the formula (I).

The preparation of the compounds according to the invention is carried out by methods known per se from the literature, as described in standard works on organic synthesis (cf., for example, T. L. Gilchrist, C. J. Moody, Chem. Rev. 77, 409 (1977); Houben-Weyl, Methoden der Organischen Chemie [Methods of Organic Chemistry], Vol. E11, p.877).

The preparation is carried out under reaction conditions which are known and suitable for the reactions mentioned. It is also possible to use variants which are known per se but not mentioned here in detail.

If desired, the starting materials can also be formed in situ, i.e. they are not isolated from the reaction mixture but immediately reacted further to give the compounds of the formula (I).

Compounds of the formula (I) in which R¹, R², R³, R⁴, R⁵, n and X are as defined under formula (I), m is 0 and Y is oxygen are prepared, for example, by reacting a carboxamide of the formula (II), in which R¹, R², n and X are as defined under formula (I) with a halogenating agent, preferably a chlorinating or brominating agent, to give a compound of the formula (III) in which R¹, R², R³, n and X are as defined under formula (I) and Z is halogen, preferably chlorine or bromine, and this compound is then reacted with a thioether R⁴R⁵S, in which R⁴ and R¹ are as defined under formula (I) in the presence of a base to give the end products of the formula (I), with the proviso that R⁴ and R⁵ are preferably not both aryl and/or heteroaryl.

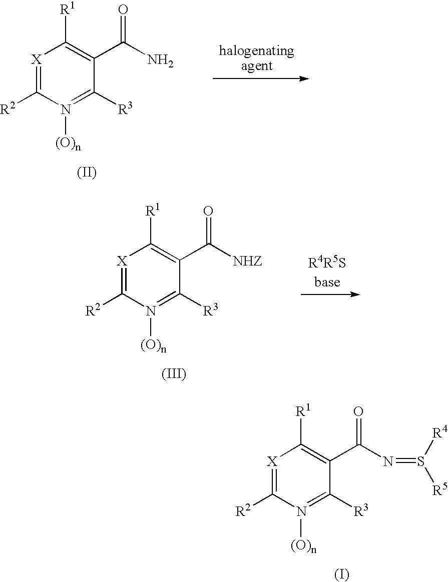

Suitable halogenating agents for preparing the compounds (Ill) are, for example, organo- or alkali metal hypochlorites, such as, for example, tert-butyl hypochlorite or sodium hypochlorite or potassium hypochlorite, alkali metal hypobromites, such as sodium hypobromite or potassium hypobromite, or the elemental halogens in the presence of a base such as, for example, alkali metal or alkaline earth metal hydroxide or carbonate.

Chlorination of amides with elemental chlorine is known in principle (Ind. J. Chem. V.35B, 1966, 1117-1118: describes the chlorination of nicotinamide with Cl₂ in 3 N HCl in two steps With a yield of 58%).

However, it is also known that the CF₃ group has a very strong electron-withdrawing effect, thus preventing electrophilic and radical attacks of the CF₃-substituted molecules. In the pyridine ring, the CF₃ group reduces basicity strongly, so that the molecule is generally not capable of forming salts.

Surprisingly, it has now been found that chlorination of 4-trifluoromethyl-nicotinamide with Cl₂ in aqueous acids (for example HCl) gives the salts of N-chloro-4-trifluoromethylnicotinamide in very good yields a high purities.

Accordingly in a further aspect of the invention there is provided a process for preparing N-chloro-4-trifluoromethylnicotinamide and salts thereof of the formula (IIIa)

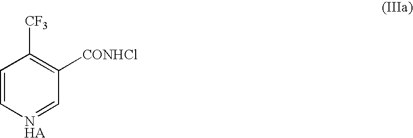

in which A is a non-oxidizable, organic or inorganic anion

by chlorination of 4-trifluoromethylnicotinamide with Cl₂ in aqueous acid and, if appropriate, subsequent anion exchange and/or, if appropriate, reaction with a base, to give N-chloro-4-trifluoromethylnicotinamide.

The starting material 4-trifluoromethylnicotinamide is known and, including its preparation, described, for example, in EP-A 0580374.

The reaction temperature is usually between −5° C. and +40° C., preferably between 0° C. and +25° C.

The process is carried out in an aqueous acid, for example HCl, H₂SO₄, HBF₄, CH₃COOH or CF₃COOH, preferably in HCl (preferred concentration 3-10% by weight). It is also possible to use mixtures of a plurality of acids.

Cl₂ is employed, preferably in gaseous form; generally in amounts of from 1 to 1.5 mole, in particular from 1 to 1.3 mole, preferably from 1 to 1.2 mole, based on 1 mole of 4-trifluoromethylnicotinamide.

The chlorination of 4-trifluoromethylnicotinamide gives the corresponding salt, preferably the hydrochloride.

Work-up is carried out by methods familiar to a person skilled in the art, for example by filtering off, washing and drying the precipitated product.

Subsequent anion exchange can be carried out by known methods which are familiar to the person skilled in the art. The salt obtained in the reaction can, for example, be dissolved in a suitable solvent in which the desired salt is insoluble. By reaction with a salt which is likewise soluble in this solvent and which contains the desired anion, the desired salt is obtained by precipitation, since it is insoluble in the chosen solvent.

If desired, the free N-chloro compound can be liberated in a simple manner familiar to the person skilled in the art, by reaction with base.

Suitable bases are, for example, hydroxides, carbonates, bicarbonates, acetates of alkali metals and alkaline earth metals, in particular those of sodium, potassium, magnesium and calcium, and furthermore tertiary amines having (C₁-C₄)-alkyl radicals. It is furthermore possible to isolate the free base by treatment with water and extraction with organic solvents.

The invention also provides salts of N-chloro-4-trifluoromethylnicotinamide of the formula (IIIa):

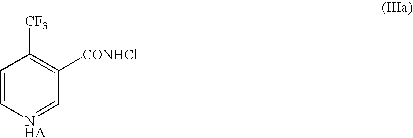

where A is a non-oxidizable, organic or inorganic anion, preferably F, HF₂, C₁, BF₄, PF₆, HSO₄, ½ SO₄, CH₃COO, CF₃COO, CF₃SO₃, CH₃SO₃, p-CH₃—C₆H₅SO₃ or H₂PO₄.

In the context of the invention, ‘non-oxidizable’ means that the corresponding anion does not react with the N—Cl group of N-chloro-4-trifluoromethyl-nicotinamide.

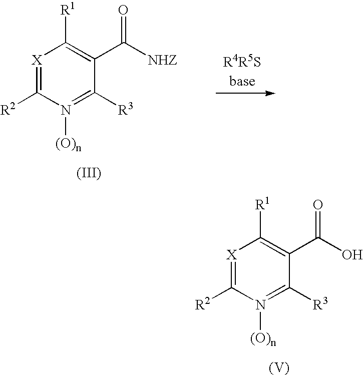

The reaction of the N-haloamides (III), optionally as salts, to the end products (I) is carried out e.g., in an inert solvent such as, for example, dichloromethane, chloroform, carbon tetrachloride or benzene, in a temperature range between 0° C. and 100° C., preferably between 20° C. and 50° C. and in the presence of a base. Suitable bases are, for example, alkali metal or alkaline earth metal hydroxides, carbonates or bicarbonates or organic bases, such as, for example, trialkylamines or pyridine.

The reaction sequence described above can, if appropriate, also be carried out as a one-pot reaction, it also being possible for intermediates of the formula (IV) in which R⁴ and R⁵ are as defined above under formula (I) and Z is a halogen radical, preferably chlorine or bromine, to occur as reaction partners of the amide (II).

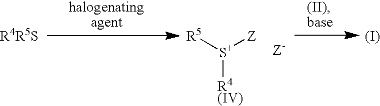

Compounds of the formula (1), in which R¹, R², R³, R⁴, R⁵, n, m and X are as defined under formula (I) and Y is oxygen can furthermore be prepared by reacting a carboxylic acid of the formula (V),

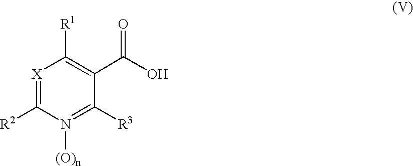

in which R¹, R², R³, X and n are as defined under formula (I) in the form of an activated derivative of this acid is reacted in the presence of a base with a compound of the formula (VI), in which R⁴, R⁵ and m are as defined under formula (1)

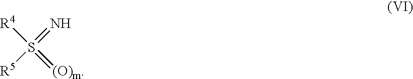

Suitable activated derivatives of the acid which may be used are, for example, anhydrides, azolides or, preferably, acid chlorides. Suitable bases are, for example, amines, such-as triethylamine, diisopropylethylamine, pyridine or lutidine or else alkali metal or alkaline earth metal hydroxides, carbonates or bicarbonates. The reaction is advantageously carried out in an inert solvent, such as, for example, dichloromethane, chloroform, carbon tetrachloride, benzene, toluene, diethyl ether or tetrahydrofuran, or else in mixtures of these solvents, in a temperature range between 0° C. and 100° C., preferably between 20° C. and 50° C.

Compounds of the Formula (Ia),

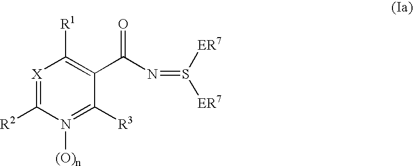

in which R¹, R², R³, R⁷, X and n are as defined in formula (I), Y is oxygen and E corresponds to an oxygen or nitrogen unit, are e.g. prepared by converting an amide of the formula (II) with a halogenating agent, advantageously tert-butyl hypochlorite, into the N,N-dichloro compound (VII), in which R¹, R², R³, n and X are as defined under formula (I)

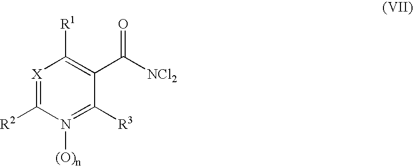

which is then converted with sulfur dichloride SCl₂ into a compound of the formula (VIII), in which R¹, R², R³, n and X are as defined under formula (I)

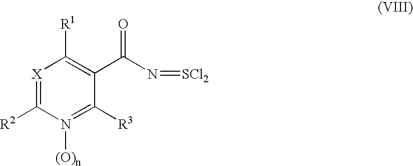

and the latter compound is reacted with at least two equivalents of a nucleophile HER, in which ER⁷ is as defined above under formula (Ia), in the presence of a base. Suitable bases are organic bases, such as, for example, triethylamine, pyridine or lutidine, or alkali metal or alkaline earth metal hydroxides, carbonates or bicarbonates and, in the case of alcohols, also alkali metal or alkaline earth metal hydrides or amides. The reaction is advantageously carried out in an inert solvent, such as, for example, dichloromethane, chloroform, carbon tetrachloride, benzene, toluene, diethyl ether or tetrahydrofuran, or in mixtures of these solvents, in a temperature range between 0° C. and 100° C., preferably between 20° C. and 50° C.

The compounds of the formula (I), in which R¹, R², R³, R⁴, R⁵, n and X are as defined under formula (I), Y is oxygen and m is 0 can furthermore be prepared by reacting an azide of the formula (IX),

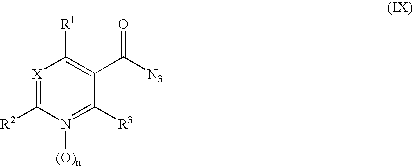

in which R¹, R², R³, X and n are as defined under formula (I) with a thioether R⁴R⁵S in which R⁴ and R⁵ are as defined under formula (I) in the presence of a catalyst, such as, for example, FeCl₂ or with irradiation, giving the end products with elimination of nitrogen.

The compounds of the formula (I), in which R¹, R², R³, n and X are as defined under formula (I), R⁴ and R⁵ are aryl radicals, Y is oxygen and m is 0 can furthermore be obtained by reacting an amide of the formula (II) with a dialkoxy-diaryl-sulfurane of the formula (X), in which R⁴ and R⁵ are aryl radicals and ORF is a fluoroalkoxy radical, preferably the 1,1,1,3,3,3-hexafluoro-2-phenyl-2-propoxy radical

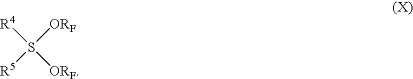

The compounds of the formula (I), in which R¹, R², R³, R⁴, R⁵, n and X are as defined under formula (I), Y is oxygen and m is 0 can furthermore be prepared by reacting a sulfoxide of the formula (XI),

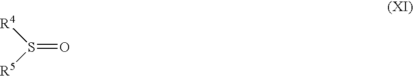

in which R⁴ and R⁵ are as defined under formula (I) in the presence of a condensing agent with an amide of the formula (II), giving the compounds of the formula (I) with elimination of water. Suitable condensing agents are, for example, phosphorus oxychloride, phosphorus(V) oxide, methanesulfonyl chloride, sulfuryl chloride, sulfur trichloride, boron trifluoride, dicyclohexylcarbodiimide, aryl cyanates or acid anhydrides, preferably trifluoroacetic anhydride or trifluoromethanesulfonic anhydride.

The compounds of the formula (I) in which n means 1 can be prepared by oxidizing the pyridine nitrogen, preferably before the introduction of the SR⁴R⁵ group (see e.g. Houben-Weyl, Methoden der Organischen Chemie, Vol. E 7b, Part 2, page 565, G. Thieme, Stuttgart 1992). Suitable oxidizing agents are, for example, organic peracids, such as 3-chloroperbenzoic acid, and H₂O₂.

If desired, the compounds of the formula (I) prepared by the above process can, if m is 0, be oxidized at the sulfur to give the compounds of the formula (I) in which m is 1. Suitable oxidizing agents are, for example, sodium periodate or organic peracids, such as 3-chloroperbenzoic acid.

Furthermore, if appropriate, compounds of the formula (I) in which R² and/or R³ are a halogen atom, preferably chlorine or fluorine, can be converted by reaction with alcohols, thiols or primary or secondary amines in the presence of a base to other compounds of the formula (I) in which the radical R² and/or R³ denotes an alkoxy, alkylthio or amino group.

Further references concerning preparation of the compounds according to the invention and the various starting materials can be found in standard works on organic synthesis, such as, for example: T. L. Gilchrist, C. J. Moody, Chem. Rev. 77, 409 (1977) or Houben-Weyl, Methoden der Organischen Chemie, Vol. E11, p.877.

Collections of compounds of the formula (I) which can be synthesized by the abovementioned scheme may also be prepared in a parallel manner, and this may be effected manually or in a semiautomated or fully automated manner. In this case, it is possible, for example, to automate the procedure of the reaction, work-up or purification of the products or of the intermediates. In total, this is to be understood as meaning a procedure as is described, for example, by S. H. DeWitt in “Annual Reports in Combinatorial Chemistry and Molecular Diversity: Automated Synthesis”, Volume 1, Verlag Escom 1997, pages 0.69 to 77.

A series of commercially available apparatuses as are offered by, for example, Stem Corporation, Woodrolfe Road, Tollesbury, Essex, CM9 8SE, England or H+P Labortechnik GmbH, Bruckmannring 28, 85764 Oberschleilβeim, Germany or Radleys, Shirehill, Saffron Walden, Essex, England, may be used for the parallel procedure of the reaction and work-up. For the parallel purification of compounds of the formula (I), or of intermediates obtained during the preparation, use may be made, inter alia, of chromatography apparatuses, for example those by ISCO, Inc., 4700 Superior Street, Lincoln, Nebr. 68504, USA.

The apparatuses mentioned lead to a modular procedure in which the individual process steps are automated, but manual operations must be performed between the process steps. This can be prevented by employing semi-integrated-or fully integrated automation systems where the automation modules in question are operated by, for example, robots. Such automation systems can be obtained, for example, from Zymark Corporation, Zymark Center, Hopkinton, Mass. 01748, USA.

In addition to what has been described here, compounds of the formula (I) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described extensively in the specialist literature, for example Barry A. Bunin in “The Combinatorial Index”, Academic Press, 1998.

The use of solid-phase-supported synthesis methods permits a series of protocols which are known from the literature and which, in turn, can be performed manually or in an automated manner. For example, the “tea-bag method” (Houghten, U.S. Pat. No. 4,631,211; Houghten et al., Proc. Natl. Acad. Sci, 1985, 82, 5131-5135), in which products by IRORI, 11149 North Torrey Pines Road, La Jolla, Calif. 92037, USA, are employed, may be semiautomated. The automation of solid-phase-supported parallel syntheses is performed successfully, for example, by apparatuses by Argonaut Technologies, Inc., 887 Industrial Road, San Carlos, Calif. 94070, USA or MultiSynTech GmbH, Wullener Feld 4, 58454 Witten, Germany.

The preparation of the processes described herein yields compounds of the formula (I) in the form of substance collections which are termed libraries. The present invention also relates to libraries which comprise at least two compounds of the formula (I).

The compounds of the formula (I) are suitable for controlling animal pests, in particular insects, arachnids, helminths and mollusks, very especially preferably for controlling insects and arachnids which are encountered in agriculture, in livestock breeding, in forests, in the protection of stored goods and materials, and in the hygiene sector, and have good plant tolerance and favorable toxicity to warm-blooded species. They are active against normally sensitive and resistant species and against all or individual developmental stages. The abovementioned pests include:

From the order of the Acarina, for example, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Eotetranychus spp., Oligonychus spp., Eutetranychus spp.

From the order of the Isopoda, for example, Oniscus aselus, Armadium vulgare, Porcellio scaber.

From the order of the Diplopoda, for example, Blaniulus guttulatus.

From the order of the Chilopoda, for example, Geophilus carpophagus, Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Blatta orientalis, Periplaneta americana, Leucophaea maderae, Blattella germanica, Acheta domesticus, Gryllotalpa spp., Locusta migratoria migratorioides, Melanoplus differentialis, Schistocerca gregaria.

From the order of the Isoptera, for example, Reticulitermes spp.

From the order of the Anoplura, for example, Phylloera vastatrix, Pemphigus spp., Pediculus humanus corporis, Haematopinus spp., Linognathus spp.

From the order of the Mallophaga, for example, Trichodectes pp., Damalinea spp.

From the order of the Thysanoptera, for example, Hercinothrips femoralis, Thrips tabaci.

From the order of the Heteroptera, for example, Eurygaster spp., Dysdercus intermedius, Piesma quadrata, Cimex lectularius, Rhodnius prolixus, Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Doralis fabae, Doralis pomi, Eriosoma lanigerum, Hyalopterus arundinis, Macrosiphum avenae, Myzus spp., Phorodon humuli, Rhopalosiphum padi, Empoasca spp., Euscelus bilobatus, Nephotettix cincticeps, Lecanium comi, Saissetia oleae, Laodelphax striatellus, Nilaparvata lugens, Aonidiella aurantii, Aspidiotus hederae, Pseudococcus spp., Psylla spp.

From the order of the Lepidoptera, for example, Pectinophora gossypiella, Bupalus piniarius, Cheimatobia brumata, Lithocolletis blancardella, Hyponomeuta padella, Plutella maculipennis, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Laphygma exigua, Mamestra brassicae, Panolis flammea, Prodenia litura, Spodoptera spp., Trichoplusia ni, Carpocapsa pomonella, Pieris spp., Chilo spp., Pyrausta nubilalis, Ephestia kuehniella, Galleria mellonella, Cacoecia podana, Capua reticulana, Choristoneura fumiferana, Clysia ambiguella, Homona magnanima, Tortrix viridana.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylloides chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrynchus assimilis, Hypera postica, Dermestes spp., Trogoderma, Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica.

From the order of the Hymenoptera, for example, Diprion spp., Hoplocampa spp., Lasius spp., Monomorium pharaonis, Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hypobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa.

From the order of the Siphonaptera, for example, Xenopsylla cheopsis, Ceratophyllus spp.

From the order of the Arachnida, for example, Scorpio maurus, Latrodectus mactans.

From the class of the helminths, for example, Haemonchus, Trichostrongulus, Ostertagia, Cooperia, Chabertia, Strongyloides, Oesophagostomum, Hyostrongulus, Ancylostoma, Ascaris and Heterakis and also Fasciola.

From the class of the Gastropoda, for example, Deroceras spp., Arion spp., Lymnaea spp., Galba spp., Succinea spp., Biomphalaria spp., Bulinus spp., Oncomelania spp.

From the class of the Bivalva, for example, Dreissena spp.

The plant-parasitic nematodes which can be controlled in accordance with the invention include, for example, the root-parasitic soil-dwelling nematodes such as, for example, those of the genera Meloidogyne (root knot nematodes, such as Meloidogyne incognita, Meloidogyne hapla and Meloidogyne javanica), Heterodera and Globodera (cyst-forming nematodes, such as Globodera rostochiensis, Globodera pallida, Heterodera trifolii) and of the genera Radopholus, such as Radopholus similis, Pratylenchus such as Pratylenchus neglectus, Pratylenchus penetrans and Pratylenchus curvitatus;

Tylenchulus such as Tylenchulus semipenetrans, Tylenchorhynchus, such as Tylenchorhynchus dubius and Tylenchorhynchus claytoni, Rotylenchus such as Rotylenchus robustus, Heliocotylenchus such as Haliocotylenchus multicinctus, Belonoaimus such as Belonoaimus longicaudatus, Longidorus such as Longidorus elongatus, Trichodorus such as Trichodorus primitivus and Xiphinerma such as Xiphinema index.

Other nematode genera which can be controlled using the compounds according to the invention are Ditylenchus (stem parasites, such as Ditylenchus dipsaci and Ditylenchus destructor), Aphelenchoides (foliar nematodes, such as Aphelenchoides ritzemabosi) and Anguina (seed nematodes, such as Anguina tritici).

The invention also relates to compositions, for example crop protection compositions, preferably insecticidal, acaricidal, ixodicidal, nematicidal, molluscicidal or fungicidal, especially preferably insecticidal and acaricidal, compositions which comprise one or more compounds of the formula (I) in addition to suitable formulation auxiliaries.

In general, the compositions according to the invention comprise 1 to 95% by weight of the active substances of the formula (I).

To prepare the compositions according to the invention, the active substance and the other additives are combined and brought into a suitable use form.

The invention also relates to compositions, in particular insecticidal and acaricidal compositions, which comprise the compounds of the formula (I) in addition to suitable formulation auxiliaries.

In general, the compositions according to the invention comprise 1 to 95% by weight of the active substances of the formula (I). They can be formulated in various ways, depending on the biological and/or chemical-physical parameters which prevail. The following are examples of possible formulations:

Wettable powders (WP), emulsifiable concentrates (EC), aqueous solutions (SL), emulsions, sprayable solutions, oil- or water-based dispersions (SC), suspoemulsions (SE), dusts (DP), seed-dressing products, granules in the form of microgranules, spray granules, coated granules and adsorption granules, water-dispersible granules (WG), ULV formulations, microcapsules, waxes or baits.

These individual types of formulations are known in principle and are described, for example, in: Winnacker-Kuichler, “Chemische Technologie” [Chemical Technology], Volume 7, C. Hauser Verlag Munich, 4^{th} Edition 1986; van Falkenberg, “Pesticides Formulations”, Marcel Dekker N.Y., 2^{nd} Ed. 1972-73; K. Martens, “Spray Drying Handbook”, 3^{rd} Ed. 1979, G. Goodwin Ltd. London.

The necessary formulation auxiliaries i.e. carrier materials and/or surface active substances such as inert materials, surfactants, solvents and other additives, are also known and described, for example, in: Watkins, “Handbook of Insecticide Dust Diluents and. Carriers”, 2^{nd} Ed., Darland Books, Caldwell N.J.; H. v. Olphen, “introduction to Clay Colloid Chemistry”, 2^{nd} Ed., J. Wiley & Sons, N.Y.; Marsden, “Solvents Guide”, 2nd Ed., Interscience, N.Y. 1950; McCutcheon's, “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte”, [Surface-active ethylene oxide adducts] Wiss. Verlagsgesell., Stuttgart 1967; Winnacker-Küchler, “Chemische Technologie”, Volume 7, C. Hanser Verlag Munich, 4^{th} Edition 1986.

Based on these formulations, it is also possible to prepare combinations with other pesticidally active materials, fertilizers and/or growth regulators, for example in the form of a ready-mix formulation or a tank mix. Wettable powders are preparations which are uniformly dispersible in water which, besides the active substance, also comprise wetters, for example polyoxyethylated alkylphenols, polyoxyethylated fatty alcohols, alkylsulfonates or alkylphenolsulfonates and dispersants, for example sodium lignosulfonate or sodium 2,2′-dinaphthylmethane-6,6′-disulfonate, in addition to a diluent or inert material.

Emulsifiable concentrates are prepared by dissolving the active substance in an organic solvent, for example butanol, cyclohexanone, dimethylformamide, xylene or else higher-boiling aromatics or hydrocarbons, with addition of one or more emulsifiers. As emulsifiers, the following can be used, for example: calcium alkylarylsulfonates such as calcium dodecylbenzenesulfonate, or nonionic emuisifiers such as fatty acid polyglycol esters, alkylaryl poiyglycol ethers, fatty alcohol polyglycol ethers, propylene oxide/ethylene oxide condensates, alkyl polyethers, sorbitan fatty acid esters, polyoxyethylene sorbitan fatty acid esters or polyoxyethylene sorbitol esters.

Dusts are obtained, for example, by grinding the active substance with finely divided solid materials, for example talc or natural clays, such as kaolin, bentonite, pyrophyllite or diatomaceous earth. Granules can be prepared either by atomizing the active substance onto adsorptive, granulated inert material or by applying active substance concentrates onto the surface of carrier materials such as sand or kaolinites, or of granulated inert material, by means of adhesives, for example polyvinyl alcohol or sodium polyacrylate, or else mineral oils. Suitable active substances can also be granulated in the manner which is customary for the preparation of fertilizer granules, if desired as a mixture with fertilizers.

The active substance concentration in wettable powders is, for example, approximately 10 to 90% by weight, the remainder to 100% by weight is composed of customary formulation auxiliaries. In the case of emulsifiable concentrates, the active substance concentration may be approximately 5 to 80% by weight. Formulations in the form of dusts usually comprise 5 to 20% by weight of active substance, sprayable solutions approximately 2 to 20% by weight. In the case of granules, the active substance content depends partly on whether the active compound is in liquid or solid form and on which granulation auxiliaries, fillers and the like are being used.

Besides, the abovementioned active substance formulations comprise, if appropriate, the tackifiers, wetters, dispersants, emulsifiers, penetrants, solvents, fillers or carriers which are conventional in each case.

For use, the concentrates, which are present in commercially available form, are, if desired, diluted in the customary manner, for example in the case of wettable powders, emulsifiable concentrates, dispersions and in some cases also microgranules, using water. Preparations in the form of dusts and granules and sprayable solutions are usually not diluted any further with other inert substances prior to use.

The application rate required varies with the external conditions such as, inter alia, temperature and humidity. It may vary within wide limits, for example between 0.0005 and 10.0 kg/ha or more of active substance, but it is preferably between 0.001 and 5 kg/ha of active compound.

The active substances according to the invention, in their commercially available formulations and in the use forms prepared from these formulations may be present in mixtures with other active substances such as insecticides, attractants, sterilants, acaricides, nematicides, fungicides, growth regulatory substances or herbicides.

The pesticides include, for example, phosphoric esters, carbamates, carboxylic esters, formamidines, tin compounds and materials produced by microorganisms.

Preferred components in mixtures are:

1. from the group of the phosphorus compounds

acephate, azamethiphos, azinphos-ethyl, azinphos-methyl, bromophos, bromophos-ethyl, cadusafos (F-67825), chlorethoxyphos, chlorfenvinphos, chlormephos, chlorpyrifos, chlorpyrifos-methyl, demeton, demeton-S-methyl, demeton-S-methyl sulfone, dialifos, diazinon, dichlorvos, dicrotophos, dimethoate, disulfoton, EPN, ethion, ethoprophos, etrimfos, famphur, fenamiphos, fenitriothion, fensulfothion, fenthion, flupyrazofos, fonofos, formothion, fosthiazate, heptenophos, isazophos, isothioate, isoxathion, malathion, methacrifos, methamidophos, methidathion, salithion, mevinphos, monocrotophos, naled, omethoate, oxydemeton-methyl, parathion, parathion-methyl, phenthoate, phorate, phosalone, phosfolan, phosphocarb (BAS-301), phosmet, phosphamidon, phoxim, pirimiphos, pirimiphos-ethyl, pirimiphos-methyl, profenofos, propaphos, proetamphos, prothiofos, pyraclofos, pyridapenthion, quinalphos, sulprofos, temephos, terbufos, tebupirimfos, tetrachlorvinphos, thiometon, triazophos, trichlorphon, vamidothion;

2. from the group of the carbamates

alanycarb (OK-135), aldicarb, 2-sec-butylphenyl methylcarbamate (BPMC), carbaryl, carbofuran, carbosulfan, cloethocarb, benfuracarb, ethiofencarb, furathiocarb, HCN-801, isoprocarb, methomyl, 5-methyl-m-cumenylbutyryl(methyl)carbamate, oxamyl, pirimicarb, propoxur, thiodicarb, thiofanox, 1-methylthio(ethylideneamino)-N-methyl-N-(morpholinothio)carbamate (UC 51717), triazamate;

3. from the group of the carboxylic esters

acrinathrin, allethrin, alphametrin, 5-benzyl-3-furylmethyl(E)-(1R)-cis-2,2-dimethyl-3-(2-oxothiolan-3-ylidenemethyl)cyclopropanecarboxylate, beta-cyfluthrin, alpha-cypermethrin, beta-cypermethrin, bioallethrin, bioallethrin ((S)-cyclopentylisomer), bioresmethrin, bifenthrin, (RS)-1-cyano-1-(6-phenoxy-2-pyridyl)methyl(1RS)-rans-3-(4-tert-butylphenyl)-2,2-dimethylcyclopropanecarboxylate (NCI 85193), cycloprothrin, cyfluthrin, cyhalothrin, cythithrin, cypermethrin, cyphenothrin, deltamethrin, empenthrin, esfenvalerate, fenfluthrin, fenpropathrin, fenvalerate, flucythrinate, flumethrin, fluvalinate (D isomer), imiprothrin (S-41311), lambda-cyhalothrin, permethrin, phenothrin® isomer), prallethrin, pyrethrins (natural products), resmethrin, tefluthrin, tetramethrin, theta-cypermethrin, tralomethrin, transfluthrin, zeta-cypermethrin (F-56701);

4. from the group of the amidines

amitraz, chlordimeform;

5. from the group of the tin compounds

cyhexatin, fenbutatin oxide;

6. others

abamectin, ABG-9008, acetamiprid, acequinocyl, Anagrapha falcitera, AKD-1022, AKD-3059, ANS-118, azadirachtin, Bacillus thuringiensis, Beauveria bassianea, bensultap, bifenazate, binapacryl, BJL-932, bromopropylate, BTG-504, BTG-505, buprofezin, camphechlor, cartap, chlorobenzilate, chlorfenapyr, chlorfluazuron, 2-(4-chlorophenyl)4,5-diphenylthiophene (UBI-T 930), chlorfentezine, chlorproxyfen, chromafenozide, clothianidine, 2-naphthylmethyl cyclopropanecarboxylate (Ro12-0470), cyromazin, diacloden (thiamethoxam), diafenthiuron, DBI-3204, ethyl 2-chloro-N-(3,5-dichloro-4-(1,1,2,3,3,3-hexafluoro-1-propyloxy)phenyl)carbamoyl)₂-carboximidate, DDT, dicofol, diflubenzuron, N-(2,3-dihydro-3-methyl-1,3-thiazol-2-ylidene)-2,4-xylidine, dihydroxymethyldihydroxypyrrolidine, dinobuton, dinocap, diofenolan, emamectin benzoate, endosulfan, ethiprole (sulfethiprole), ethofenprox, etoxazole, fenazaquin, fenoxycarb, fipronil, fluazuron, flumite (flufenzine, SZI-1.21), 2-fluoro-5-(4-(4-ethoxyphenyl)₄-methyl-1-pentyl)diphenyl ether (MTI 800), granulosis and nuclear polyhedrosis viruses, fenpyroximate, fenthiocarb, fluacrypyrim, flubenzimine, flubrocythrinate, flucycloxuron, flufenoxuron, flufenzine, flufenprox, fluproxyfen, gamma-HCH, halfenozide, halofenprox, hexaflumuron (DE_{—}473), hexythiazox, HOI-9004, hydramethylnon (AC 217300), IKI-220, indoxacarb, ivermectin, L-14165, imidacloprid, indoxacarb (DPX-MP062), kanemite (AKD-2023), lufenuron, M-020, M-020, methoxyfenozide, milbemectin, NC-196, neemgard, nidinoterfuran, nitenpyram, 2-nitromethyl-4,5-dihydro-6H-thiazine (DS 52618), 2-nitromethyl-3,4-dihydrothiazole (SD 35651), 2-nitromethylene-1,2-thiazinan-3-ylcarbamaldehyde (WL 108477), novaluron, pirydaryl, propargite, protrifenbute, pymethrozine, pyridaben, pyrimidifen, pyriproxyfen, NC-196, NC—I 111, NNI-9768, novaluron (MCW-275), OK-9701, OK-9601, OK-9602, OK-9802, R-195, RH-0345, RH-2485, RYI-210, S-1283, S-1833, SI-8601, silafluofen, silomadine (CG-177), spinosad, spirodiclofen, SU-9118, tebufenozide, tebufenpyrad, teflubenzuron, tetradifon, tetrasul, thiacloprid, thiocyclam, thiamethoxam, tolfenpyrad, triazamate, triethoxyspinosyn A, triflumuron, verbutin, vertalec (mykotal), YI-5301.

The abovementioned components for combinations are known active substances, many of which are described in Ch. R Worthing, S. B. Walker, The Pesticide Manual, 12′ Edition, British Crop Protection Council, Famham 2000.

The active substance content of the use forms prepared from the commercially available formulations may range from 0.00000001 up to 95% by weight of active substance, preferably between 0.00001 and 1% by weight.

Application is effected in a customary manner adapted to suit the use forms.

The active substances according to the invention are also suitable for controlling endoparasites and ectoparasites in the veterinary medicine sector and/or in the field of animal keeping. The active substances according to the invention are applied in the known manner, such as by oral administration in the form of, for example, tablets, capsules, drinks or granules, by dermal application in the form of, for example, dipping, spraying, pouring on and spotting on, and dusting, and by parenteral administration in the form of, for example, an injection.

Accordingly, the compounds of the formula (I) according to the invention can also be employed particularly advantageously in livestock keeping (for example cattle, sheep, pigs and poultry such as chickens, geese and the like). In a preferred embodiment of the invention, the compounds, if appropriate in suitable formulations, are administered orally to the animals, if appropriate together with the drinking water or feed. Since excretion in the feces is efficient, the development of insects in the animals' feces can be prevented very easily in this manner. The dosages and formulations which are suitable in each case depend, in particular, on the species and the developmental stage of the productive livestock and also on the risk of infestation and can be determined readily and established by customary methods. For example, the compounds can be employed in cattle at dosages of 0.01 to 1 mg/kg bodyweight.

In addition to the abovementioned application methods, the active compounds of the formula (I) according to the invention have excellent systemic action. Accordingly, the active compounds can also be introduced into the plants via parts of the plant, both below ground and above ground (for example root, stolons, stem, trunk, leaf), if the active compounds are applied, in liquid or solid form, onto the plant and/or onto the direct vicinity of the plant (for example granules in soil application, application in flooded rice paddies, trunk injection in the case of trees, stem bandages in the case of perennial plants).

Furthermore, the active compounds according to the invention, optionally in coformulation with fungicides, are particularly suitable for the treatment of vegetative and generative plant propagation material, such as, for example, of seeds, for example of cereals, vegetables, cotton, rice, sugar beet and other crops and ornamental plants, of bulbs, seedlings and tubers of other crops and ornamental plants which are propagated vegetatively. The treatment can be carried out before sowing or before planting (for example by special seed coating techniques, by dressing in liquid or solid form or as a seed box treatment), during sowing or planting or after sowing or planting by special application techniques (for example furrow treatment). The amount of active compound used can vary within a relatively large range, depending on the application. In general, the application rates are between 1 g and 10 kg of active compound per hectare of soil surface. The treatment methods for plant propagation material and the plant propagation material treated in this manner are also provided by the invention.

The compounds of the formula (I) can also be employed for controlling harmful organisms in crops of known genetically engineered plants or genetically engineered plants yet to be developed. As a rule, the transgenic plants are distinguished by especially advantageous properties, for example by resistances to particular crop protection agents, resistances to plant diseases or pathogens of plant diseases, such as particular insects or microorganisms such as fungi, bacteria or viruses. Other particular properties concern, for example, the harvested material with regard to quantity, quality, storage properties, composition and specific constituents. Thus, transgenic plants are known where the starch content is increased, or the starch quality is altered, or where the harvested material has a different fatty acid composition.

The use in economically important transgenic crops of useful plants and ornamentals is preferred, for example of cereals such as wheat, barley, rye, oats, millet, rice, cassaya and maize or else crops of sugar beet, cotton, soya, oilseed rape, potatoes, tomatoes, peas and other types of vegetables.

When used in transgenic crops, in particular those which have resistances to insects, effects are frequently observed, in addition to the effects against harmful organisms to be observed in other crops, which are specific for application in the transgenic crop in question, for example an altered or specifically widened spectrum of pests which can be controlled, or altered application rates which may be employed for application.

The invention therefore also relates to the use of compounds of the formula (I) for controlling harmful organisms in transgenic crop plants.

The use of the compounds according to the invention embraces, in addition to direct application onto the pests, any other application in which compounds of the formula (I) act on the pests. Such indirect applications can, for example, be the use of compounds which, for example in the soil, the plant or the pest, decompose into compounds of the formula (I) or are degraded into compounds of the formula (I).

In addition to their lethal effect on pests, the compounds of the formula (I) are also distinguished by a pronounced repellant effect.

A repellant for the purposes of the invention is a substance or substance mixture which has a warding-off or fending-off effect on other live organisms, in particular harmful pests and nuisance pests. The term also encompasses effects such as the antifeeding effect, where the intake of feed is disturbed or prevented (antifeedant effect), supression of oviposition, or an effect on the development of the population.

The invention therefore also relates to the use of compounds of the formula (I) for achieving the abovementioned effects, in particular in the case of the pests stated in the biological examples.

The invention also relates to a method of repelling harmful organisms, where one or more compounds of the formula (I) are applied to the site from which the harmful organisms are to be fended off or warded off.

In the case of a plant, application may mean, for example, a treatment of the plant, but also of the seed.

As regards the effect on populations, it is interesting to note that effects can also be observed in succession during the development of a population, where summation may take place. In such a case, the individual effect itself may only have an efficacy of markedly less than 100% but in total an efficacy of 100% is still achieved in the end.

Moreover, the compounds of the formula (I) are distinguished by the fact that the composition is usually applied earlier than in the case of a direct control, if the abovementioned effects are to be exploited. The effect frequently lasts over a long period, so that a duration of action of over 2 months is achieved.

The effects are not only found in insects, but also in spider mites and molluscs.

The content of the German Patent Applications 10014006.8 and 10057911.6, whose priority the present application claims, and the appended abstract is incorporated herein by reference.

The examples below serve to illustrate the invention.

### A. CHEMICAL EXAMPLES

Preparation of Starting Materials

Preparation of N-chloro-4-trifluoromethylnicotinamides (compounds (III))

1.a) A mixture of 0.01 mol of 4-trifluoromethylnicotinamide or 2,6-dichloro-4-trifluoromethylnicotinamide and 0.012 mol of tert.-butyl hypochlorite in 20 ml of CCl₄ was heated at 80° C. (water bath) for 2 hours. The reaction mixture was cooled to 1 s-2⁰° C. and the precipitated N-chloroamides were then filtered off, washed with 10 ml of CCl₄ and dried at 40-50° C. under reduced pressure (10-15 mm Hg).

N-Chloro-4-trifluoromethylnicotinamide: Yield 85%, m.p. 136-138° C.

N-Chloro-2,6-dichloro-4-trifluoromethylnicotinamide: Yield 80%, m.p. 160-161° C.

1.b) 4-Trifluoromethyl-N-chloronicotinamide hydrochloride.

In a 0.5 I four-necked flask fitted with a stirrer with glass shaft and bearing, thermometer, gas inlet tube and condenser, 150 g of 5% HCl and 50 g of 4-trifluoromethylnicotinamide were initially charged, the mixture was stirred at room temperature for 15 min and 20 g of Cl₂ were then introduced from the gas bottle such that the chlorine was taken up virtually completely. After addition of about 10 g of chlorine, the starting material dissolved and the product precipitated as a white precipitate. Crystalline product was filtered off with suction and the mother liquor was kept separately. The product was washed once with 30-40 ml of ice-cold water and dried. This gave 58 g of product, 92% yield (m.p. (decomposition) 150° C., active chlorine 13.7%, HCl 13.5%). The product reacted with NaHCO₃ with formation of N-chloro-4-trifluoromethylnicotinamide of m.p. 140-141° C.

1.c) 4-Trifluoromethyl-N-chloronicotinamide hydrotetrafluoroborate.

10 g of N-chloro-4-trifluoromethylnicotinamide hydrochloride were dissolved in 30 ml of ethanol, and the solution of 10 [lacuna] of NaBF₄ in 10 ml of HBF₄ was added. The mixture was stirred for 20 min, and crystalline product was filtered off with suction, washed once with 30-40 ml of ice-cold water and dried. This gave 12 g of 4-trifluoromethyl-N-chloronicotinamide hydrotetrafluoroborate.

2. Synthesis of sulfoxylic acid amides (R₂NNR₂)

At 0-5° C., a solution of 0.02 mol of SCl₂ in 20 ml of dry diethyl ether was added dropwise with stirring to a solution of 0.08 mol of the secondary amine in 50 ml of dry diethyl ether.

The reaction mixture was stirred at 20-25° C. for 1 hour, the precipitated amine hydrochloride was filtered off and the filtrate was concentrated under reduced pressure (10-15 mm Hg, 25-30° C.). The dialkylamides of sulfoxylic acid are oils which were purified by vacuum distillation (except for the bisamylamide, which was not purified any further).

Me₂N—S—NMe₂₁ yield 78%, bp. 35-40° C. (30 mm Hg).

Et₂N—S—NEt₂, yield 70%, bp. 88-90° C. (15 mm Hg).

(i-Pr₂)N—S—N(i-Pr)₂, yield 68%, bp. 105-108° C. (15 mm Hg).

(n-Bu₂)N—S—N(n-Bu)₂, yield 85%, bp. 118-120° C. (0.1 mm Hg).

Ph(Et)N—S—NPh(Et), yield 70%, pp. 185-188° C. (0.04 mm Hg).

(n-Am₂)N—S—N(n-Am)₂, yield 76%.

3. Synthesis of N,N-dichloroamides of 4-trifluoromethylnicotinic acid and N,N-dichloroamides of 2,6-dichloro-4-trifluoromethylnicotinic acid (compounds (VII))

A mixture of 0.02 mol of 4-trifluoromethylnicotinamide or 2,6-dichloro-4-trifluoromethylnicotinamide, 0.044 mol of t-butyl hypochlorite and 20 ml of CCl₄ was heated at 70° C. for 2 hours. The solvent and t-butanol were removed under reduced pressure (10-15 mm Hg, 60° C.). The resulting N,N-dichloroamides are yellow oils which were used without any further purification.

4-Trifluoromethylnicotinic acid derivative: yield 95%, ¹⁹F-NMR (CCl₄): −63.25 2,6-Dichloro-4-trifluoromethylnicotinic acid derivative: yield 92%, ¹⁹F-NMR (CCl₄): -63.82

4. Synthesis of N-3-(4-trifluoromethyl)pyridoylimidosulfurous acid dichlorides (compounds (VII))

For this reaction, all solvents have to be anhydrous. At 0-5° C., a solution of 0.02 mol of the N,N-dichloroamides described above in 10 ml of CCl₄ was added with stirring to a solution of 0.02 mol of sulfur dichloride in 20 ml of CCl₄. The reaction mixture was stirred at 0-5° C. for 15 minutes and then at 20-25° C. for 2 hours. A small amount of precipitate was filtered off and the filtrate was concentrated under reduced pressure at 10-15 mm Hg and 50-60° C. The products are-slightly brown oils and were reacted without any further purification.

4-Trifluoromethylnicotinic acid derivative: yield 93%, ¹⁹F-NMR (CCl₄): −62.69 2,6-Dichloro-4-trifluoromethylnicotinic acid derivative: yield 94%, ¹⁹F-NMR (CCl₄): -61.42

Preparation of the End Products

At 20-25° C., a solution of 0.01 mol of dialkyl sulfide, alkyl aryl sulfide or sulfoxylic acid diamide and 0.01 mol of triethylamine in 10 ml of benzene was added dropwise to a solution of 0.01 mol of N-chloronicotinamide in 15 ml of benzene or acetonitrile. The reaction mixture was stirred at 20-25° C. for 3 hours, the precipitated triethylamine hydrochloride was filtered off and the filtrate was concentrated at 40-50° C. under reduced pressure (10-15 mm Hg) and, if required, chromatographed on silica gel.

This gave, for example, examples 1, 2, 3, 4, 5, 7, 8, 9, 11, 12, 16, 19 and 203.

Ex. No. 66

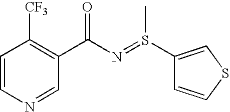

0.50 g (0.0015 mol) of methyl-(3-thienylysulfimide-(2,4,6-trimethylbenzenesulfonate) and 0.38 g (0.0018 mol) of 4-trifluoromethylnicotinyl chloride were dissolved in 20 ml of dichloromethane and, at 0° C., a solution of 0.38 g (0.0038 mol) of triethylamine in 5 ml of dichloromethane was added dropwise. The mixture was stirred at room temperature for 1.5 days and extracted with saturated sodium chloride solution, and the organic phase was dried. Concentration gave a colorless solid which was purified further by silica gel chromatography. This gave 390 mg (81.6% of theory) of product as a colorless solid.

M.p. 94-95° C.

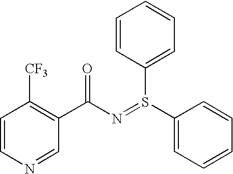

Example No. 464

0.87 g (1.2 mmol) of S,S-bis-[α,α-bis-(trifluoromethan)benzenemethanolato]diphenylsulfur (Martin sulfurane dehydrating agent, Aldrich) and 0.25 g (1.3 mmol) of 4-trifluoromethylnicotinamide were stirred at room temperature for 6 hours. The solution was allowed to stand overnight and then concentrated, and the residue was stirred with heptane. The undissolved solid (unreacted amide) was filtered off and the residue was chromatographed over silica gel (ethyl acetate/heptane 9:1). This gave 0.12 g (25.6% of theory) of product as a colorless solid.

M.p. 102-103° C.

Conversion of the Compounds (VIII) into the End Products

At 5-10° C., a solution of 0.061 mol of dialkylamine in 50 ml of benzene was added to a solution of 0.015 mol of the dichlorosulfimides (VIII) described above in 30 ml of benzene (in the case of dimethylamine, dimethylamine gas was passed through the solution). The reaction mixture was stirred at 20-25° C. for 20 hours and the precipitated amine hydrochloride was filtered off. The filtrate was concentrated under reduced pressure (10-15 mm Hg at 50-60° C.) and the end product was purified by recrystallization or washing with hexane.

Examples 5, 6, 10, 29, 30 and 31 were obtained in this manner.

Synthesis of N-(2-amino-6-chloro-4-trifluoromethylnicotinoyl)sulfimides

A mixture of 0.01 mol of a (2,6-dichloro-4-trifluoromethylnicotinoyl)sulfimide and 0.02 mol of the corresponding amine in 15 ml of benzene was heated at 80° C. for 3 hours. In the case of dimethylamine, the reaction was carried out by bubbling the gaseous amine at 20-25° C. through the solution. The hydrochloride of the amine was filtered off and the filtrate was concentrated under reduced pressure (10-15 mm Hg at 50-60° C.). The end product was washed with hexane and purified by recrystallization.

Examples 33, 35- and 36 were obtained in this manner.

Synthesis of Sulfoximides

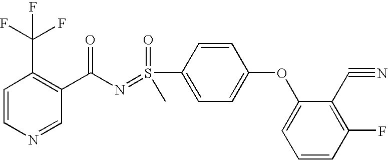

Example 718

500 mg (1.7 mmol) of [4-(2-cyano-3-fluorophenoxy)phenyl]methylsulfoximine and 210 mg (2.0 mmol) of triethylamine were initially charged in 20 ml of dichloromethane, and, at 0° C., a solution of 430 mg (2.0 mmol) of 4-trifluoromethylnicotinoyl chloride was added dropwise. The mixture was stirred at room temperature for 6 hours and then concentrated. The residue was taken up in water/dichloromethane, the organic phase was dried and the solvent was removed. Further purification was carried out by silica gel chromatography (ethyl acetate/heptane 9:1). This gave a resin which crystallized on tritruration with heptane.

Yield: 420 mg of colorless crystals (53.3% of theory)

M.p.: 100-102° C.

Example 700

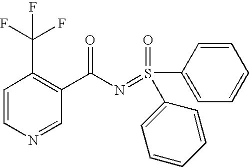

At 0° C., a solution of 170 mg (0.98 mmol) of 3-chloroperbenzoic acid was added dropwise to a solution of 270 mg (0.75 mmol) of (4-trifluoromethylnicotinoyl)diphenyl-sulfimide in 10 ml of dichloromethane, and the mixture was stirred at room temperature. The mixture was allowed to stand overnight and then extracted twice with sodium bicarbonate solution, and the organic phase was dried and concentrated. Further purification was carried out by silica gel chromatography (ethyl acetate). This gives 120 mg (41% of theory) of product as a colorless oil.

TABLE 1

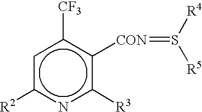

| Example | R² | R³ | S R⁴ R⁵ | m.p. [° C.] |
|---|---|---|---|---|
| 1 | H | H | S(CH₃)₂ | 86-90 |
| 2 | H | H | 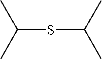 | 87-89 |
| 3 | H | H | S(C₂H₅)₂ | 52-53 |
| 4 | H | H | 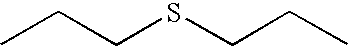 | Oil |
| 5 | H | H | S[N(CH₃)₂]₂ | 87-89 |
| 6 | H | H | S[N(C₂H₅)₂]₂ | 40-45 |
| 7 | H | H | 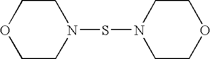 | 86-89 |
| 8 | H | H | 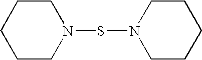 | 99-101 |
| 9 | H | H | 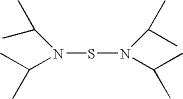 | 101-102 |
| 10 | H | H | 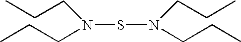 | 61-62 |
| 11 | H | H | 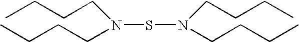 | Oil |
| 12 | H | H | 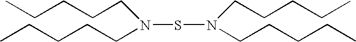 | Oil |
| 13 | H | H | 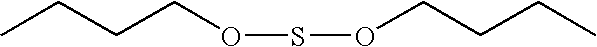 | Oil |
| 14 | H | H | 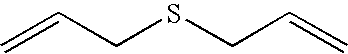 | Oil |
| 15 | H | H | 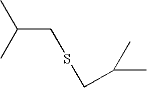 | 64-65 |
| 16 | H | H | 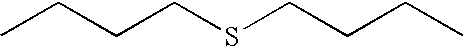 | Oil |
| 17 | H | H | (C₆H₅CH₂)₂S | 104-105 |
| 18 | H | H | C₂H₅O—S—OC₂H₅ | Oil |
| 19 | H | H | 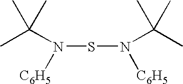 | Oil |
| 20 | H | H | 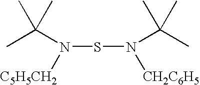 | 156-157 |
| 21 | H | H | 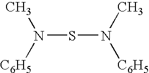 | 80-81 |
| 22 | H | H | 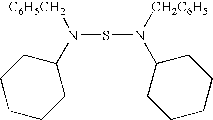 | 101 |
| 23 | H | H | 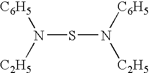 | Oil |
| 24 | H | H | 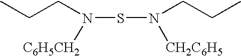 | Oil |
| 25 | H | H | 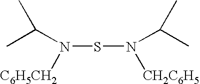 | Oil |
| 26 | H | H | 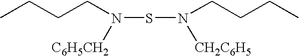 | 61-62 |
| 27 | H | H | 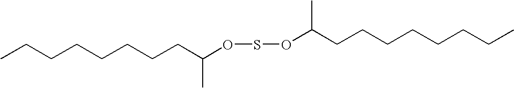 | Oil |
| 28 | H | H | 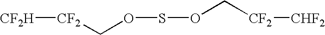 | Oil |
| 29 | Cl | Cl | 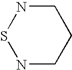 | 130-132 |
| 30 | Cl | Cl | 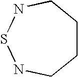 | 78-80 |
| 31 | Cl | Cl | 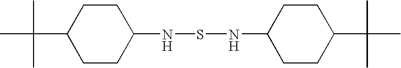 | Resin |
| 32 | Cl | Cl | 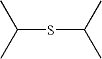 | 129-130 |
| 33 | Cl | N(C₂H₅)₂ | 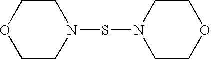 | 152-154 |
| 34 | Cl | Cl | (CH₃)₂N—S—N(CH₃)₂ | 90-91 |
| 35 | Cl | N(CH₃)₂ | (CH₃)₂N—S—N(CH₃)₂ | 81-83 |
| 36 | Cl | 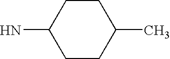 | 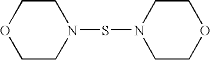 | 125-127 |
| 37 | H | H | S[N(i-C₃H₇)₂]₂ | 101-102 |
| 38 | H | H | CH₃SC₂H₅ | |
| 39 | H | H | 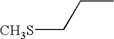 | |
| 40 | H | H | 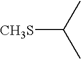 | 72 |
| 41 | H | H | 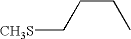 | |
| 42 | H | H | 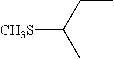 | Oil |
| 43 | H | H | 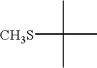 | |
| 44 | H | H | 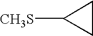 | |
| 45 | H | H | 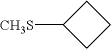 | |
| 46 | H | H | 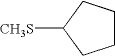 | |
| 47 | H | H | 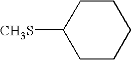 | |
| 48 | H | H | 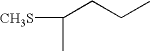 | |
| 50 | H | H | 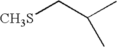 | |
| 51 | H | H | 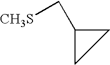 | |
| 52 | H | H | CH₃SCH₂CF₃ | |
| 53 | H | H | CH₃SC₆H₅ | |
| 54 | H | H | 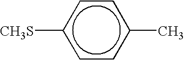 | 105 |
| 55 | H | H | 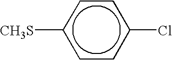 | Oil |
| 56 | H | H | 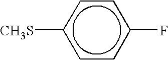 | |
| 57 | H | H | 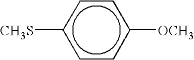 | Oil |
| 58 | H | H | 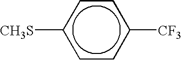 | |
| 59 | H | H | 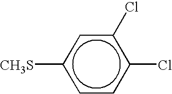 | |
| 60 | H | H | 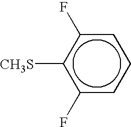 | |
| 61 | H | H | 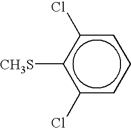 | |
| 62 | H | H | 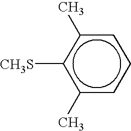 | |
| 63 | H | H | 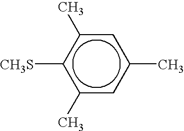 | |
| 64 | H | H | 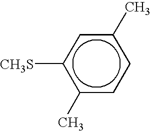 | |
| 65 | H | H | 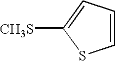 | |
| 66 | H | H | 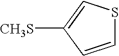 | 94-95 |
| 67 | H | H | 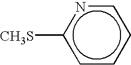 | |
| 68 | H | H | 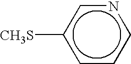 | |
| 69 | H | H | 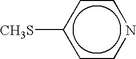 | |
| 70 | H | H | 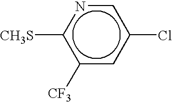 | |
| 71 | H | H | CH₃SCH₂C₆H₅ | |
| 72 | H | H | 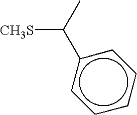 | |
| 73 | H | H | 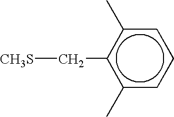 | |
| 74 | H | H | 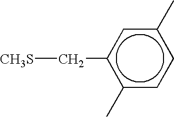 | 138-140 |
| 75 | H | H | 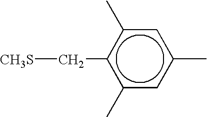 | |
| 76 | H | H | 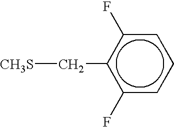 | |
| 77 | H | H | 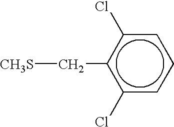 | |
| 78 | H | H | 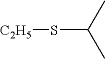 | |
| 79 | H | H | 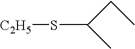 | |
| 80 | H | H | C₂H₅—S—CH₂CF₃ | |
| 81 | H | H | 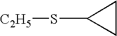 | |
| 82 | H | H | 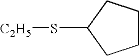 | |
| 83 | H | H | 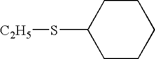 | |
| 84 | H | H | C₂H₅—S—C₆H₅ | oil |
| 85 | H | H | 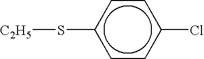 | |
| 86 | H | H | 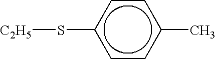 | |
| 87 | H | H | 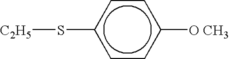 | oil |
| 88 | H | H | 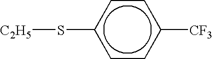 | |
| 89 | H | H | 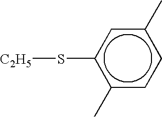 | 94-102 |
| 90 | H | H | C₂H₅S—CH₂C₆H₅ | oil |
| 91 | H | H | 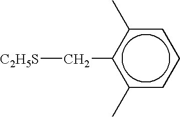 | |
| 92 | H | H | 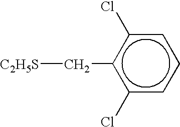 | |
| 93 | H | H | 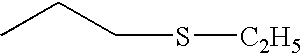 | |
| 94 | H | H | 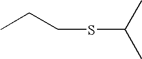 | 60 |
| 95 | H | H | 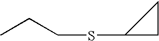 | |
| 96 | H | H | 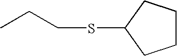 | |
| 97 | H | H | 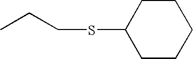 | resin |
| 98 | H | H | 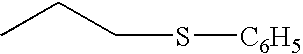 | |
| 99 | H | H | 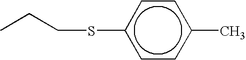 | |
| 100 | H | H | 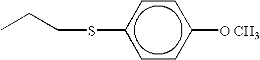 | resin |
| 101 | H | H | 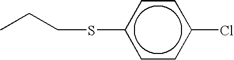 | |
| 102 | H | H | 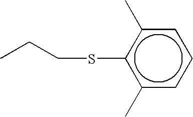 | |
| 103 | H | H | 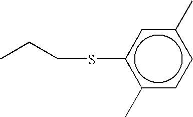 | |
| 104 | H | H | 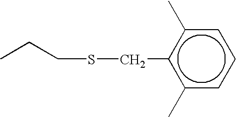 | |
| 105 | H | H | 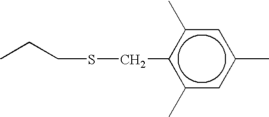 | |
| 106 | H | H | 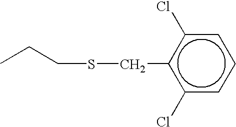 | |
| 107 | H | H | 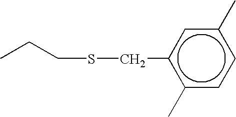 | |
| 108 | H | H | 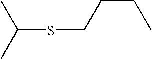 | oil |
| 109 | H | H | 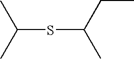 | oil |
| 110 | H | H | 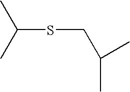 | oil |
| 111 | H | H | 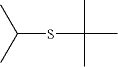 | |
| 112 | H | H | 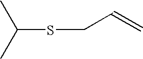 | |
| 113 | H | H | 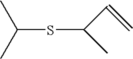 | |
| 114 | H | H | 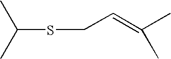 | oil |
| 115 | H | H | 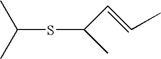 | |
| 116 | H | H | 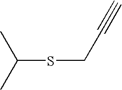 | |
| 117 | H | H | 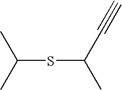 | |
| 118 | H | H | 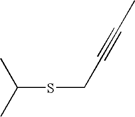 | |
| 119 | H | H | 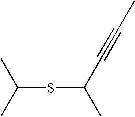 | |
| 120 | H | H | 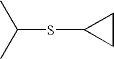 | oil |
| 121 | H | H | 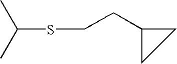 | 84 |
| 122 | H | H | 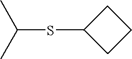 | resin |
| 123 | H | H | 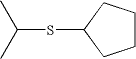 | resin |
| 124 | H | H | 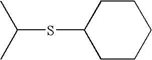 | oil |
| 125 | H | H | 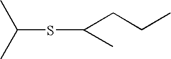 | |
| 126 | H | H | 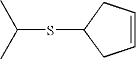 | |
| 127 | H | H | 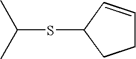 | |
| 128 | H | H | 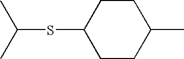 | |
| 129 | H | H | 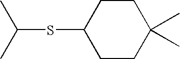 | |
| 130 | H | H | 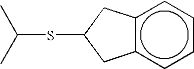 | |
| 131 | H | H | 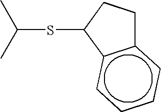 | |
| 132 | H | H | 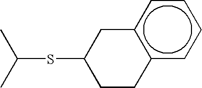 | |
| 133 | H | H | 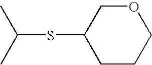 | |
| 134 | H | H | 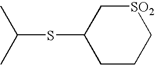 | |
| 135 | H | H | 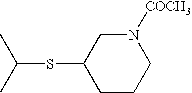 | |
| 136 | H | H | 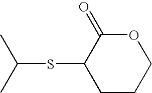 | |
| 137 | H | H | 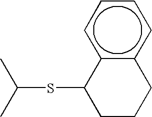 | |
| 138 | H | H | 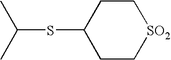 | |
| 139 | H | H | 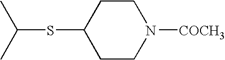 | |
| 140 | H | H | 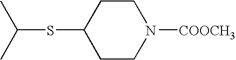 | |
| 141 | H | H | 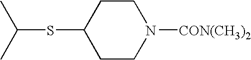 | |
| 142 | H | H | 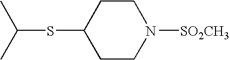 | |
| 143 | H | H | 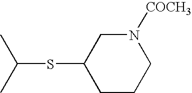 | |
| 144 | H | H | 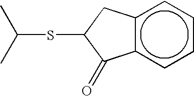 | |
| 145 | H | H | 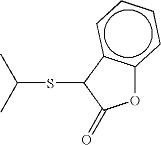 | |
| 146 | H | H | 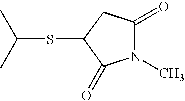 | |
| 147 | H | H | 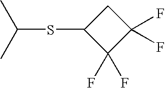 | |
| 148 | H | H | 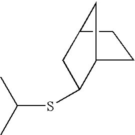 | |
| 149 | H | H | 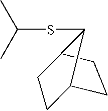 | |
| 150 | H | H | 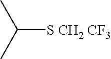 | |
| 151 | H | H | 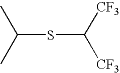 | |
| 152 | H | H | 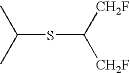 | |
| 153 | H | H | 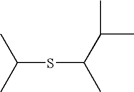 | |
| 154 | H | H | 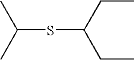 | |
| 155 | H | H | 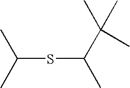 | |
| 156 | H | H | 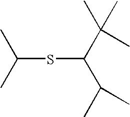 | |
| 157 | H | H | 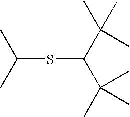 | |
| 158 | H | H | 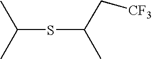 | |
| 159 | H | H | 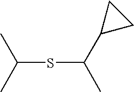 | |
| 160 | H | H | 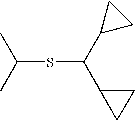 | |
| 161 | H | H | 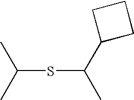 | |
| 162 | H | H | 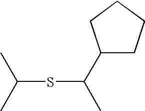 | |
| 163 | H | H | 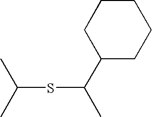 | |
| 164 | H | H | 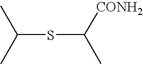 | |
| 165 | H | H | 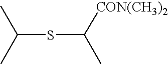 | |
| 166 | H | H | 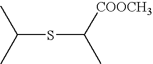 | |
| 167 | H | H | 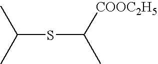 | |
| 168 | H | H | 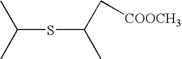 | |
| 169 | H | H | 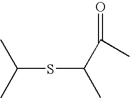 | |
| 170 | H | H | 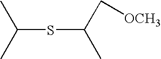 | |
| 171 | H | H | 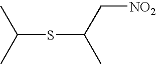 | |
| 172 | H | H | 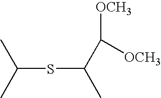 | |
| 173 | H | H | 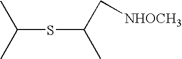 | |
| 174 | H | H | 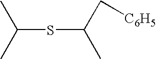 | |
| 175 | H | H | 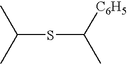 | 96 |
| 176 | H | H | 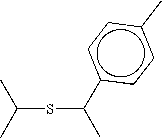 | |
| 177 | H | H | 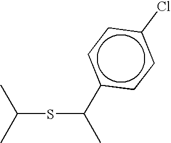 | |
| 178 | H | H | 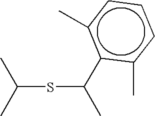 | |
| 179 | H | H | 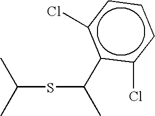 | |
| 180 | H | H | 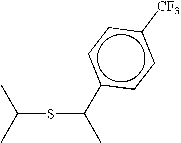 | |
| 181 | H | H | 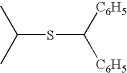 | oil |
| 182 | H | H | 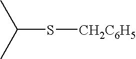 | resin |
| 183 | H | H | 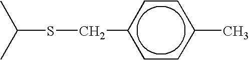 | |
| 184 | H | H | 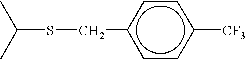 | 170 |
| 185 | H | H | 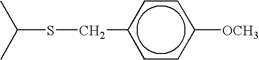 | 108 |
| 186 | H | H | 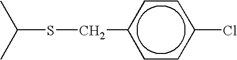 | |
| 187 | H | H | 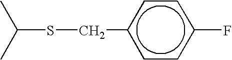 | |
| 188 | H | H | 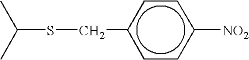 | 168 |
| 189 | H | H | 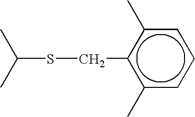 | |
| 190 | H | H | 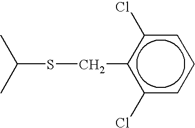 | |
| 191 | H | H | 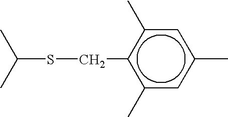 | 163 |
| 192 | H | H | 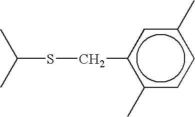 | |
| 193 | H | H | 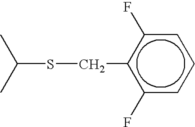 | 71 |
| 194 | H | H | 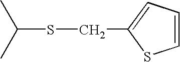 | |
| 195 | H | H | 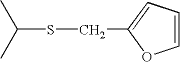 | 77 |
| 196 | H | H | 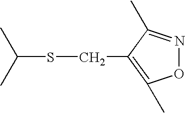 | oil |
| 197 | H | H | 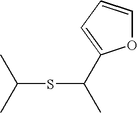 | |
| 198 | H | H | 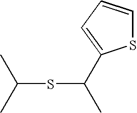 | |
| 199 | H | H | 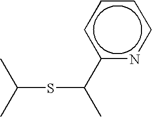 | |
| 200 | H | H | 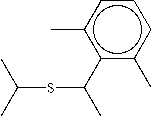 | |
| 201 | H | H | 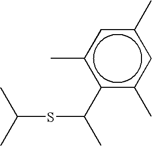 | |
| 202 | H | H | 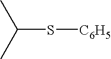 | oil |
| 203 | H | H | 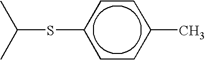 | oil |
| 204 | H | H | 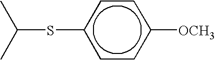 | 120-121 |
| 205 | H | H | 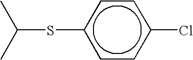 | |
| 206 | H | H | 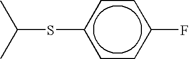 | oil |
| 207 | H | H | 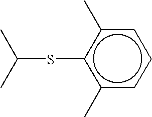 | |
| 208 | H | H | 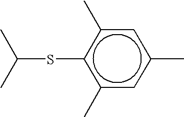 | |
| 209 | H | H | 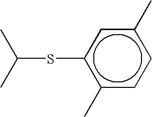 | 109-110 |
| 210 | H | H | 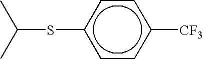 | |
| 211 | H | H | 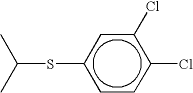 | |
| 212 | H | H | 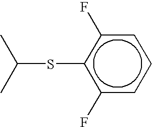 | |
| 213 | H | H | 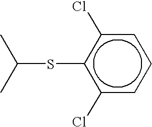 | |
| 214 | H | H | 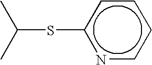 | 72-73 |
| 215 | H | H | 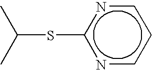 | |
| 216 | H | H | 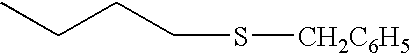 | oil |
| 217 | H | H | 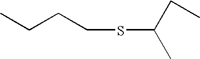 | oil |
| 218 | H | H | 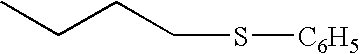 | oil |
| 219 | H | H | 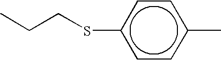 | oil |
| 220 | H | H | 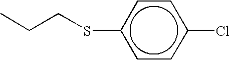 | 88-91 |
| 221 | H | H | 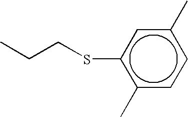 | oil |
| 222 | H | H | 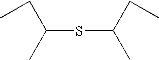 | oil |
| 223 | H | H | 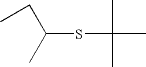 | oil |
| 224 | H | H | 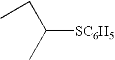 | |
| 225 | H | H | 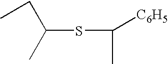 | oil |
| 226 | H | H | 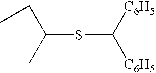 | oil |
| 227 | H | H | 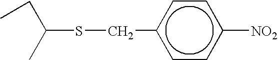 | 128 |
| 228 | H | H | 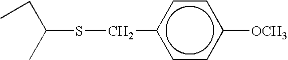 | 65 |
| 229 | H | H | 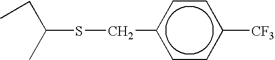 | 140 |
| 230 | H | H | 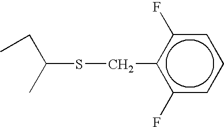 | oil |
| 231 | H | H | 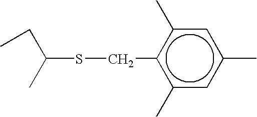 | 132 |
| 232 | H | H | 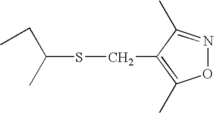 | 125 |
| 233 | H | H | 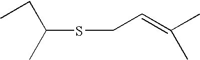 | oil |
| 234 | H | H | 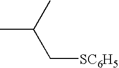 | oil |
| 235 | H | H | 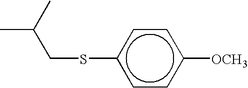 | 122-123 |
| 236 | H | H | 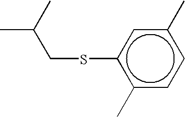 | 98-101 |
| 237 | H | H | 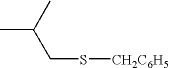 | oil |
| 239 | H | H | 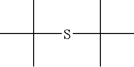 | |
| 240 | H | H | 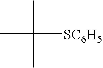 | |
| 241 | H | H | 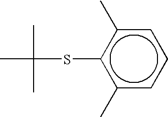 | |
| 242 | H | H | 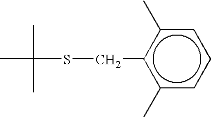 | |
| 243 | H | H | 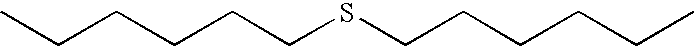 | oil |
| 244 | H | H | 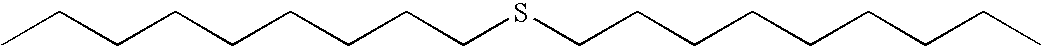 | oil |
| 245 | H | H | 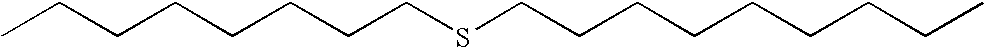 | oil |
| 246 | H | H | 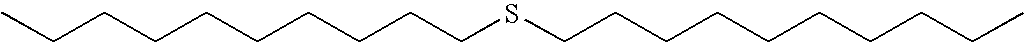 | oil |
| 247 | H | H | 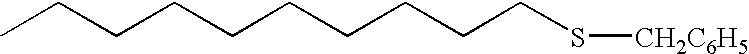 | oil |
| 248 | H | H | 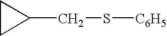 | 94-97 |
| 249 | H | H | 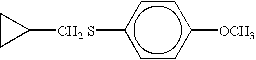 | oil |
| 250 | H | H | 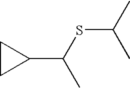 | |
| 251 | H | H | 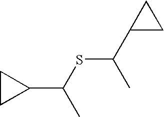 | |
| 252 | H | H | 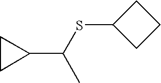 | |
| 253 | H | H | 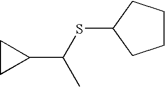 | |
| 254 | H | H | 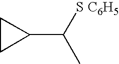 | |
| 256 | H | H | 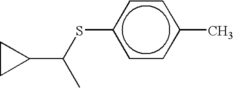 | |
| 257 | H | H | 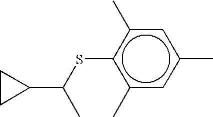 | |
| 258 | H | H | 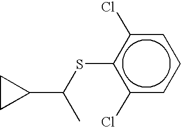 | |
| 259 | H | H | 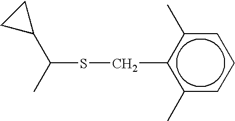 | |
| 260 | H | H | 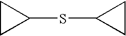 | |
| 261 | H | H | 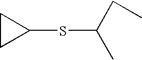 | |
| 262 | H | H | 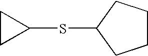 | |
| 263 | H | H | 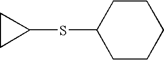 | |
| 264 | H | H | 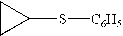 | |
| 265 | H | H | 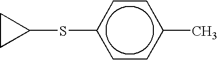 | |
| 266 | H | H | 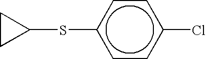 | |
| 267 | H | H | 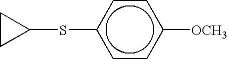 | |
| 268 | H | H | 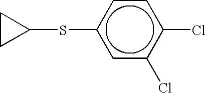 | |
| 269 | H | H | 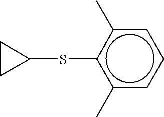 | |
| 270 | H | H | 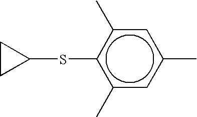 | |
| 271 | H | H | 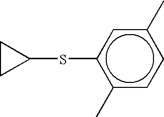 | |
| 272 | H | H | 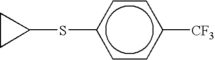 | |
| 273 | H | H | 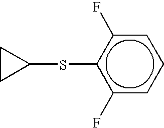 | |
| 274 | H | H | 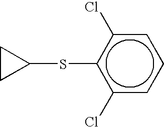 | |
| 275 | H | H | 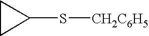 | |
| 276 | H | H | 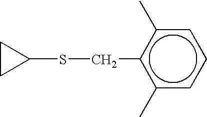 | |
| 277 | H | H | 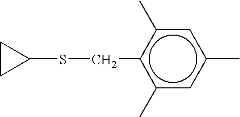 | |
| 278 | H | H | 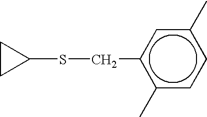 | |
| 279 | H | H | 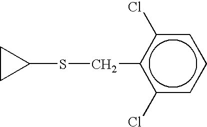 | |
| 280 | H | H | 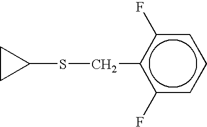 | |
| 281 | H | H | 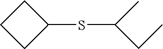 | |
| 282 | H | H | 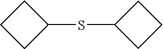 | |
| 283 | H | H | 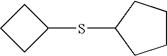 | |
| 284 | H | H | 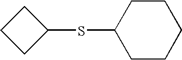 | |
| 285 | H | H | 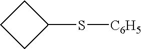 | |
| 286 | H | H | 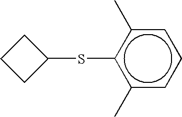 | |
| 287 | H | H | 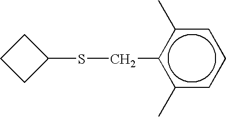 | |
| 288 | H | H | 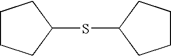 | oil |
| 289 | H | H | 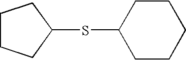 | oil |
| 290 | H | H | 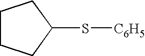 | |
| 291 | H | H | 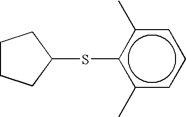 | |
| 292 | H | H | 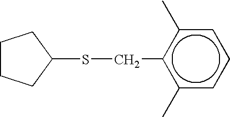 | |
| 293 | H | H | 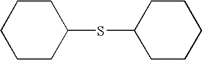 | |
| 294 | H | H | 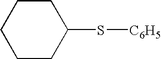 | |
| 294 | H | H | 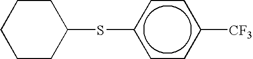 | resin |
| 295 | H | H | 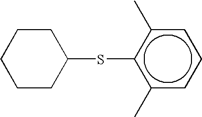 | |
| 296 | H | H | 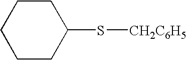 | resin |
| 297 | H | H | 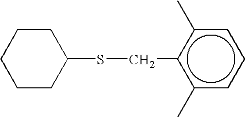 | |
| 298 | H | H | 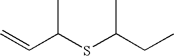 | |
| 299 | H | H | 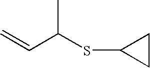 | |
| 300 | H | H | 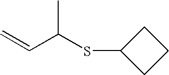 | |
| 301 | H | H | 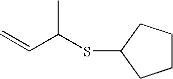 | |
| 302 | H | H | 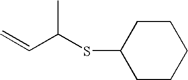 | |
| 303 | H | H | 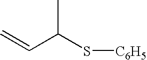 | |
| 304 | H | H | 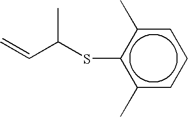 | |
| 305 | H | H | 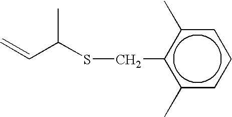 | |
| 306 | H | H | 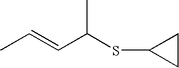 | |
| 307 | H | H | 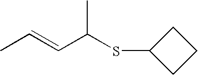 | |
| 308 | H | H | 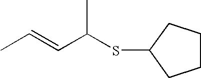 | |
| 309 | H | H | 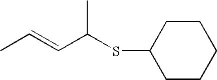 | |
| 310 | H | H | 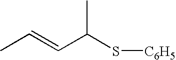 | |
| 311 | H | H | 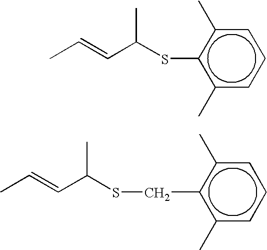 | |
| 312 | H | H | 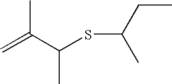 | |
| 313 | H | H | 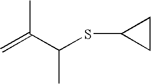 | |
| 314 | H | H | 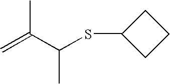 | |
| 315 | H | H | 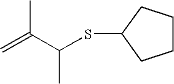 | |
| 316 | H | H | 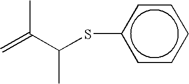 | |
| 317 | H | H | 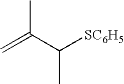 | |
| 318 | H | H | 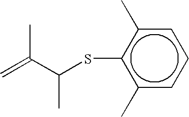 | |
| 319 | H | H | 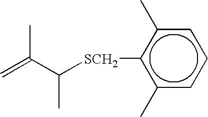 | |
| 320 | H | H | 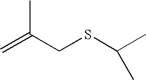 | |
| 321 | H | H | 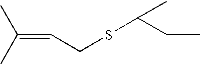 | oil |
| 322 | H | H | 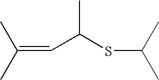 | |
| 323 | H | H | 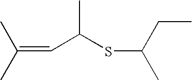 | |
| 324 | H | H | 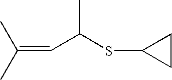 | |
| 325 | H | H | 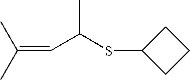 | |
| 326 | H | H | 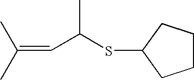 | |
| 327 | H | H | 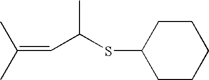 | |
| 328 | H | H | 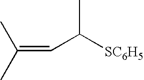 | |
| 329 | H | H | 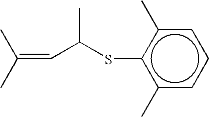 | |
| 330 | H | H | 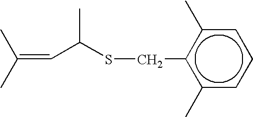 | |
| 331 | H | H | 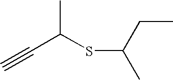 | |
| 332 | H | H | 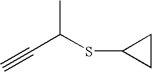 | |
| 333 | H | H | 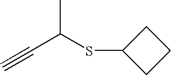 | |
| 334 | H | H | 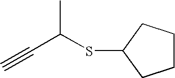 | |
| 335 | H | H | 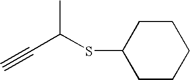 | |
| 336 | H | H | 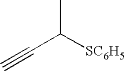 | |
| 337 | H | H | 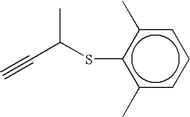 | |
| 338 | H | H | 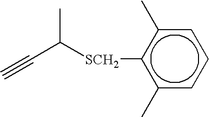 | |
| 339 | H | H | 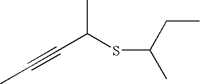 | |
| 340 | H | H | 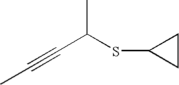 | |
| 341 | H | H | 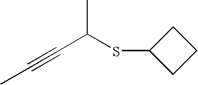 | |
| 342 | H | H | 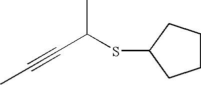 | |
| 343 | H | H | 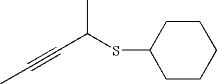 | |
| 344 | H | H | 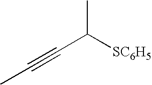 | |
| 345 | H | H | 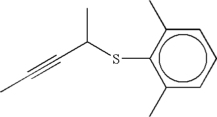 | |
| 346 | H | H | 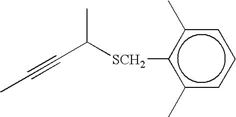 | |
| 347 | H | H | 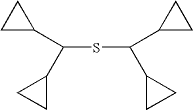 | |
| 348 | H | H | 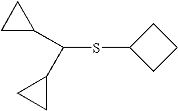 | |
| 349 | H | H | 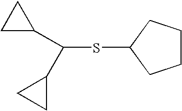 | |
| 350 | H | H | 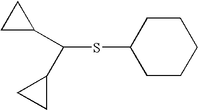 | |
| 351 | H | H | 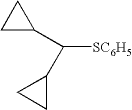 | |
| 352 | H | H | 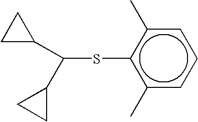 | |
| 353 | H | H | 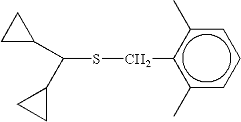 | |
| 354 | H | H | 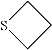 | |
| 355 | H | H | 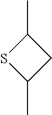 | |
| 356 | H | H | 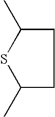 | resin |
| 357 | H | H | 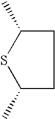 | resin |
| 358 | H | H | 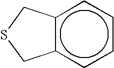 | |
| 359 | H | H | 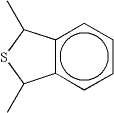 | resin |
| 360 | H | H | 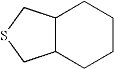 | |
| 361 | H | H | 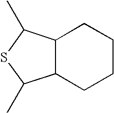 | |
| 362 | H | H | 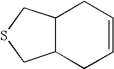 | |
| 363 | H | H | 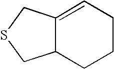 | |
| 364 | H | H | 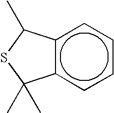 | |
| 365 | H | H | 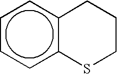 | |
| 366 | H | H | 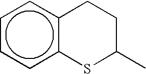 | |
| 367 | H | H | 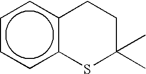 | |
| 368 | H | H | 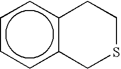 | |
| 369 | H | H | 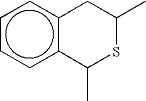 | |
| 370 | H | H | 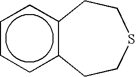 | |
| 371 | H | H | 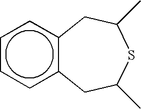 | |
| 372 | H | H | 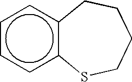 | |
| 373 | H | H | 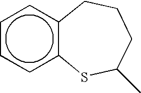 | |
| 374 | H | H | 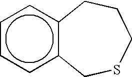 | |
| 375 | H | H | 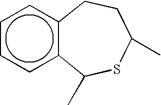 | |
| 376 | H | H | 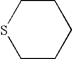 | 121 |
| 377 | H | H | 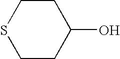 | |
| 378 | H | H | 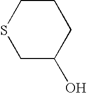 | resin |
| 379 | H | H | 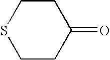 | resin |
| 380 | H | H | 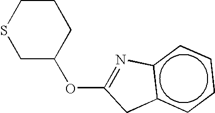 | resin |
| 381 | H | H | 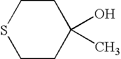 | |
| 382 | H | H | 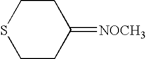 | |
| 383 | H | H | 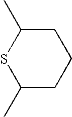 | |
| 384 | H | H | 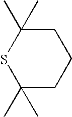 | |
| 385 | H | H | 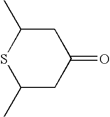 | |
| 386 | H | H | 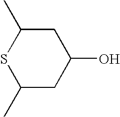 | |
| 387 | H | H | 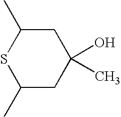 | |
| 388 | H | H | 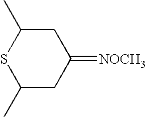 | |
| 389 | H | H | 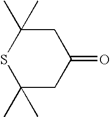 | |
| 390 | H | H | 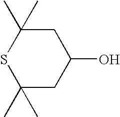 | |
| 391 | H | H | 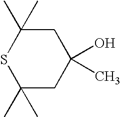 | |
| 392 | H | H | 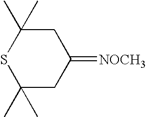 | |
| 393 | H | H | 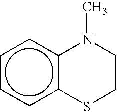 | |
| 394 | H | H | 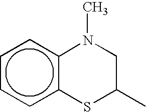 | |
| 395 | H | H | 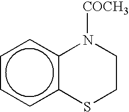 | |
| 396 | H | H | 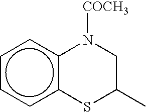 | |
| 397 | H | H | 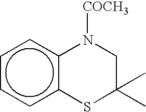 | |
| 398 | H | H | 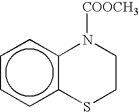 | |
| 399 | H | H | 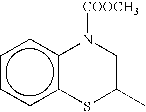 | |
| 400 | H | H | 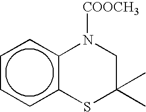 | |
| 401 | H | H | 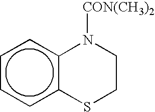 | |
| 402 | H | H | 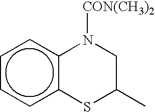 | |
| 403 | H | H | 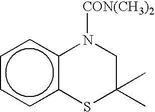 | |
| 404 | H | H | 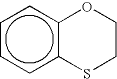 | |
| 405 | H | H | 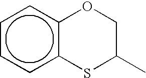 | |
| 406 | H | H | 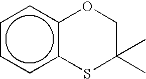 | |
| 407 | H | H | 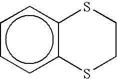 | |
| 408 | H | H | 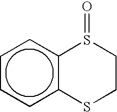 | |
| 409 | H | H | 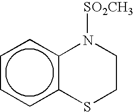 | |
| 410 | H | H | 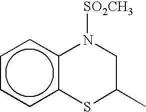 | |
| 411 | H | H | 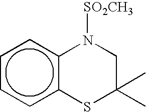 | |
| 412 | H | H | 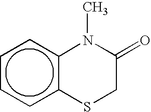 | |
| 413 | H | H | 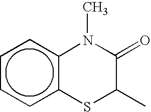 | |
| 414 | H | H | 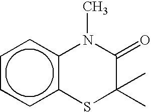 | |
| 415 | H | H | 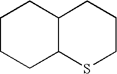 | |
| 416 | H | H | 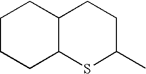 | |
| 417 | H | H | 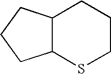 | |
| 418 | H | H | 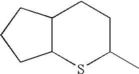 | |
| 419 | H | H | 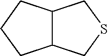 | |
| 420 | H | H | 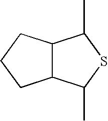 | |
| 421 | H | H | 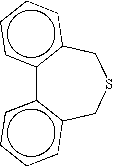 | resin |
| 422 | H | H | 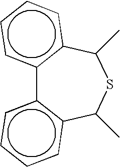 | |
| 423 | H | H | 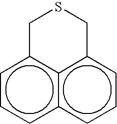 | |
| 424 | H | H | 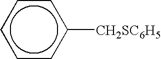 | |
| 425 | H | H | 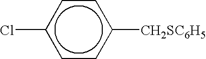 | |
| 426 | H | H | 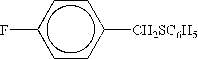 | |
| 427 | H | H | 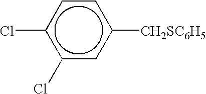 | |
| 428 | H | H | 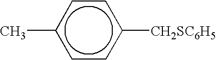 | |
| 429 | H | H | 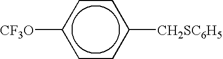 | 104 |
| 430 | H | H | 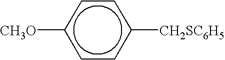 | |
| 431 | H | H | 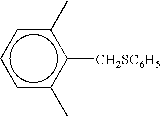 | |
| 432 | H | H | 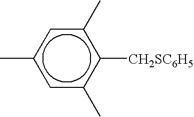 | |
| 433 | H | H | 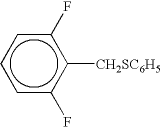 | 130 |
| 434 | H | H | 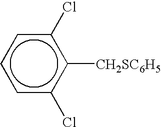 | |
| 435 | H | H | 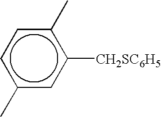 | |
| 436 | H | H | C₆H₅CH₂SCH₂C₆H₅ | |
| 437 | H | H | 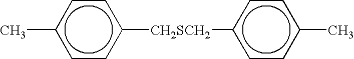 | |
| 438 | H | H | 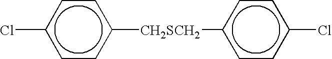 | |
| 439 | H | H | 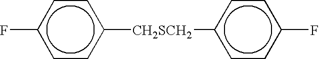 | |
| 440 | H | H | 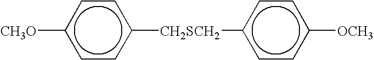 | |
| 441 | H | H | 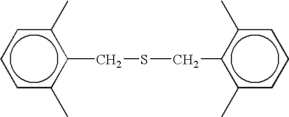 | |
| 442 | H | H | 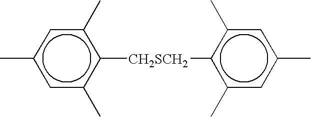 | |
| 443 | H | H | 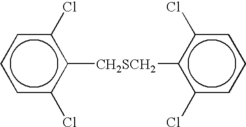 | |
| 444 | H | H | 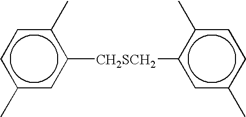 | |
| 445 | H | H | 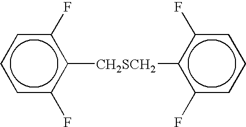 | |
| 446 | H | H | 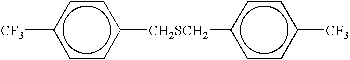 | |
| 447 | H | H | 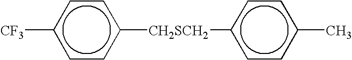 | |
| 448 | H | H | 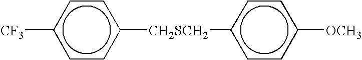 | |
| 449 | H | H | 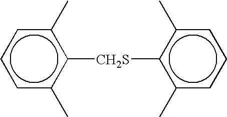 | |
| 450 | H | H | 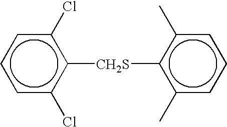 | |
| 451 | H | H | 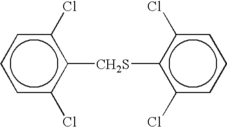 | |
| 452 | H | H | 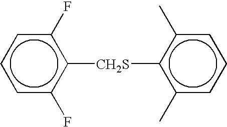 | |
| 453 | H | H | 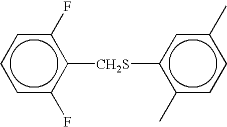 | 154 |
| 454 | H | H | 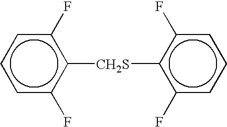 | |
| 455 | H | H | 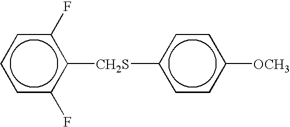 | 146 |
| 456 | H | H | 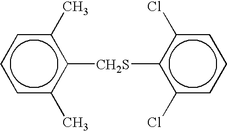 | |
| 457 | H | H | 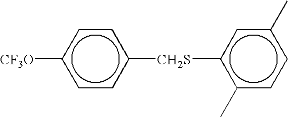 | 140 |
| 458 | H | H | 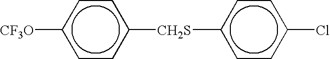 | 123 |
| 459 | H | H | 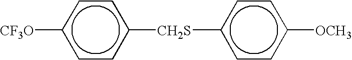 | resin |
| 460 | H | H | 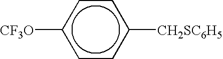 | 104 |
| 461 | H | H | 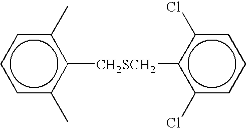 | |
| 462 | H | H | 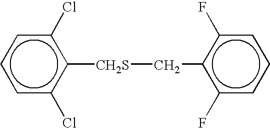 | |
| 463 | H | H | 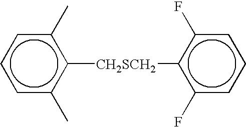 | |
| 464 | H | H | C₆H₅SC₆H₅ | 101-102 |
| 465 | H | H | 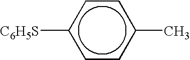 | |
| 466 | H | H | 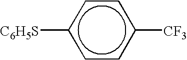 | |
| 467 | H | H | 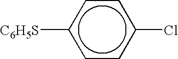 | |
| 468 | H | H | 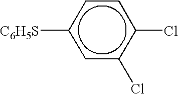 | |
| 469 | H | H | 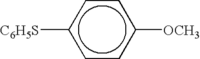 | |
| 470 | H | H | 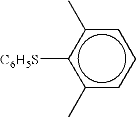 | |
| 471 | H | H | 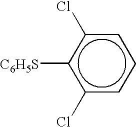 | |
| 472 | H | H | 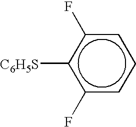 | |
| 473 | H | H | 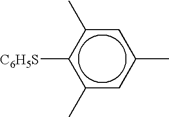 | |
| 474 | H | H | 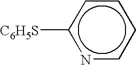 | |
| 475 | H | H | 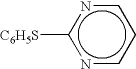 | |
| 476 | H | H | 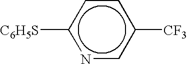 | |
| 477 | H | H | 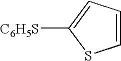 | 78-80 |
| 478 | H | H | 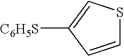 | 83-84 |
| 479 | H | H | 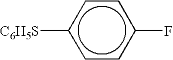 | |
| 480 | H | H | 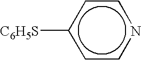 | |
| 481 | H | H | 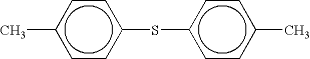 | |
| 482 | H | H | 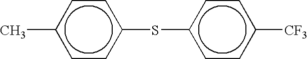 | |
| 483 | H | H | 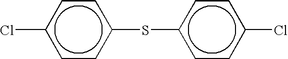 | |
| 484 | H | H | 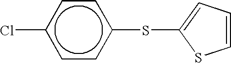 | resin |
| 485 | H | H | 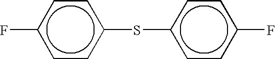 | |
| 486 | H | H | 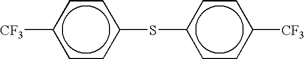 | |
| 487 | H | H | 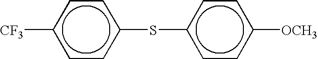 | |
| 488 | H | H | 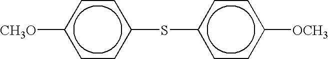 | |
| 489 | H | H | 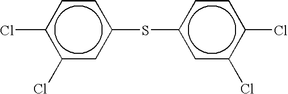 | |
| 490 | H | H | 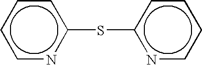 | |
| 491 | H | H | 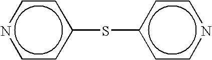 | |
| 492 | H | H | 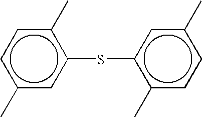 | |
| 493 | H | H | 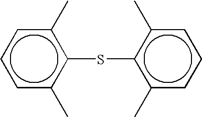 | |
| 494 | H | H | 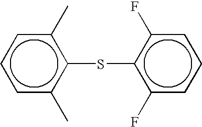 | |
| 495 | H | H | 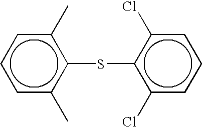 | |
| 496 | H | H | 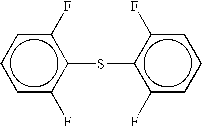 | |
| 497 | H | H | 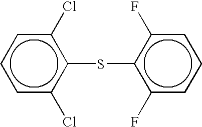 | |
| 498 | H | H | 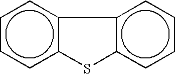 | 178-179 |
| 499 | H | H | 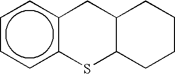 | |
| 500 | H | H | 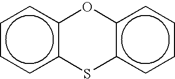 | 206-207 |
| 501 | H | H | 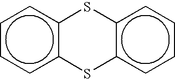 | 163-164 |
| 502 | H | H | 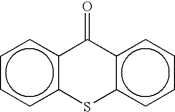 | |
| 503 | H | H | 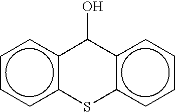 | |
| 504 | H | H | 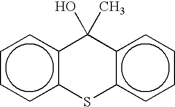 | |
| 505 | H | H | 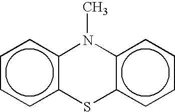 | 172-173 |
| 506 | H | H | 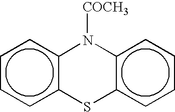 | |
| 507 | H | H | 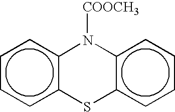 | |
| 508 | H | H | 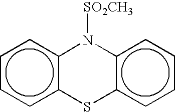 | |
| 509 | H | H | 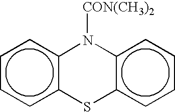 | |
| 510 | H | H | 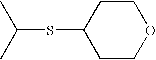 | |
| 511 | H | H | 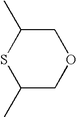 | |
| 512 | H | H | 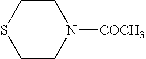 | |
| 513 | H | H | 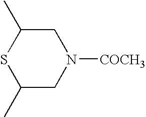 | |
| 514 | H | H | 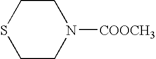 | |
| 515 | H | H | 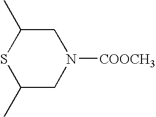 | |
| 516 | H | H | 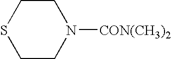 | |
| 517 | H | H | 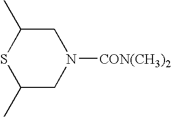 | |
| 518 | H | H | 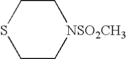 | |
| 519 | H | H | 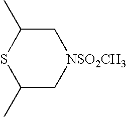 | |
| 520 | H | H | 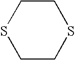 | |
| 521 | H | H | 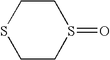 | |
| 522 | H | H | 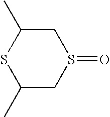 | |
| 523 | H | H | 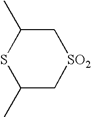 | |
| 524 | H | H | 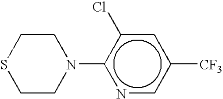 | |
| 525 | H | H | 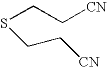 | resin |
| 526 | H | H | 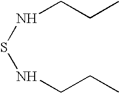 | 61-62 |
| 527 | Cl | Cl | 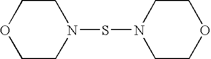 | 163-164 |
| 528 | H | H | 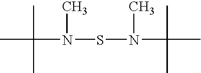 | |
| 529 | H | H | 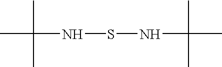 | |
| 530 | H | H | 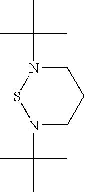 | |
| 531 | H | H | 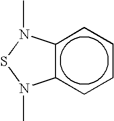 | |
| 532 | H | H | 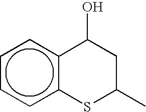 | |
| 533 | H | H | 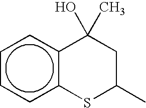 | |
| 534 | H | H | 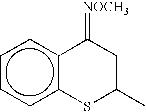 | |
| 535 | H | H | 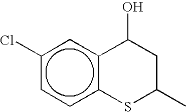 | |
| 536 | H | H | 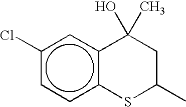 | |
| 537 | H | H | 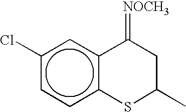 | |
| 538 | H | H | 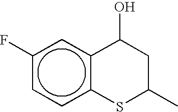 | |
| 539 | H | H | 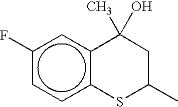 | |
| 540 | H | H | 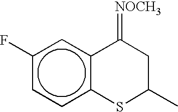 | |
| 541 | H | H | 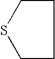 | resin |
| 542 | H | H | 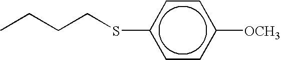 | resin |
| 543 | H | H | 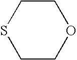 | resin |
| 544 | H | H | 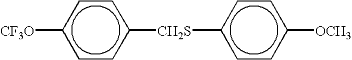 | resin |
| 545 | H | H | 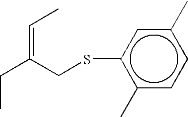 | 123-126 |

TABLE 2

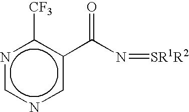

| Example | SR⁴R⁵ | m.p. [° C.] |
|---|---|---|
| 600 | 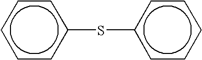 | |
| 601 | 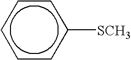 | |
| 602 | 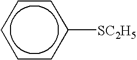 | |
| 603 | 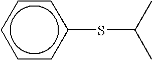 | |
| 604 | 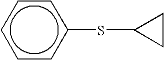 | |
| 605 | 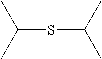 | |
| 606 | 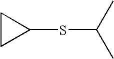 | |
| 607 | 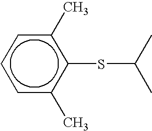 | |
| 608 | 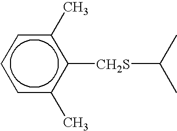 | |
| 609 | 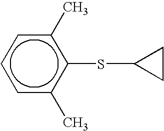 | |
| 610 | 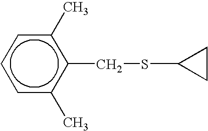 | |
| 612 | 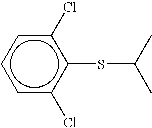 | |
| 613 | 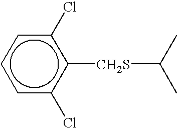 | |
| 614 | 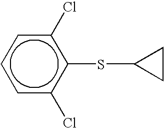 | |
| 615 | 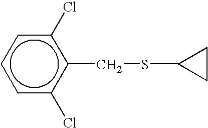 | |

All of the sulfimide derivatives (m=0) listed in tables 1 and 2 can also be present as the corresponding sulfoximides (m=1). Some examples of representatives of this class of substances are listed in table 5.

TABLE 3

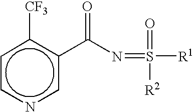

| Example | 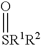 | m.p. [° C.] |
|---|---|---|
| 700 | 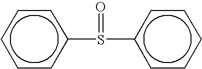 | Resin |
| 701 | 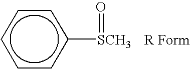 | Resin |
| 702 | 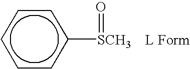 | Resin |
| 703 | 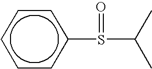 | |
| 704 | 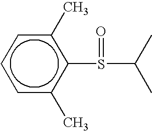 | |
| 705 | 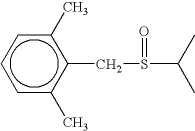 | |
| 706 | 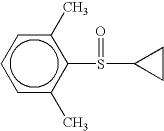 | |
| 707 | 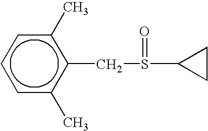 | |
| 708 | 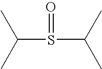 | 90 |
| 709 | 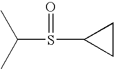 | |
| 710 | 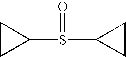 | |
| 711 | 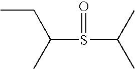 | |
| 712 | 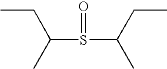 | oil |
| 713 | 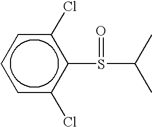 | |
| 714 | 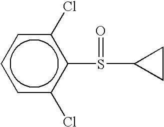 | |
| 715 | 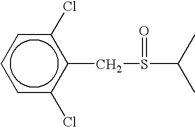 | |
| 716 | 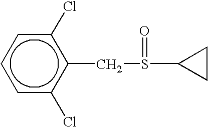 | |
| 717 | 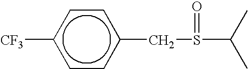 | 181 |
| 718 | 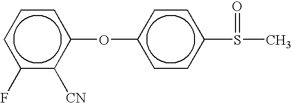 | 100-102 |
| 719 | 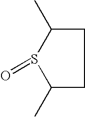 | resin |

### B. FORMULATION EXAMPLES

a) A dust is obtained by mixing 10 parts by weight of active substance and 90 parts by weight of talc as inert material and comminuting the mixture in a hammer mill.

b) A wettable powder which is readily dispersible in water is obtained by mixing 25 parts by weight of active substance, 65 parts by weight of kaolin-containing quartz as inert material, 10 parts by weight of potassium lignosulfonate and 1 part by weight of sodium oleoylmethyltaurinate as wetter and dispersant and grinding the mixture in a pinned-disk mill.

c) A dispersion concentrate which is readily dispersible in water is prepared by mixing 40 parts by weight of active substance with 7 parts by weight of a sulfosuccinic monoester, 2 parts by weight of a sodium lignosulfonate and 51 parts by weight of water and grinding the mixture in a ball mill to a fineness of below 5 microns.

d) An emulsifiable concentrate can be prepared from 15 parts by weight of active substance, 75 parts by weight of cyclohexane as solvent and 10 parts by weight of oxyethylated nonylphenol (10 EO) as emulsifier.

e) Granules can be prepared from 2 to 15 parts by weight of active substance and an inert granule carrier material such as attapulgite, pumice granules and/or quartz sand. It is expedient to use a suspension of the wettable powder of Example b) with a solids content of 30%, which is sprayed onto the surface of attapulgite granules, and these are dried and mixed intimately. The wettable powder amounts to approx. 5% by weight and the inert carrier material to approx. 95% by weight of the finished granules.

### C. BIOLOGICAL EXAMPLES

### Example 1

Germinated field bean seeds (Vicia faba) with seed roots were transferred into brown glass bottles filled with tap water and then populated with about 100 black bean aphids (Aphis fabae). Plants and aphids were then dipped into an aqueous solution of the formulated preparation to be examined for 5 seconds. After they had drained, plants and animals were stored in a climatized chamber (16 hours of light/day, 25° C., 40-60% relative atmospheric humidity). After 3 and 6 days of storage, the effect of the preparation on the aphids was determined. At a concentration of 300 ppm (based on the content of active compound), the preparations of Example Nos. 1, 2, 3, 4, 5, 6, 7, 8, 10, 11, 12, 13, 14, 15, 16, 17, 18, 20, 21, 22, 23, 24, 25, 27, 28, 37, 40, 42, 53, 54, 55, 57, 64, 66, 84, 87, 89, 90, 94, 97, 108, 110, 114, 120, 121, 122, 123, 175, 181, 182, 183, 184, 185, 191, 193, 195, 196, 202, 204, 209, 217, 218, 220, 221, 222, 223, 225, 226, 227, 228, 229, 230, 231, 232, 233, 234, 235, 237, 243, 244, 245, 246, 249, 264, 356, 357, 359, 376, 378, 379, 380, 429, 453, 455, 458, 464, 478, 484, 500, 539, 540, 541, 542, 543, 708, 709, 712, 717, 718 and 719 caused a mortality of 90-100% among the aphids.

### Example 2

Germinated field bean seeds (Vicia faba) with seed roots were transferred into brown glass bottles filled with tap water. Four milliliters of an aqueous solution of the formulated preparation to be examined were pipetted into the brown glass bottle. The field bean was then heavily populated with about 100 black bean aphids (Aphis fabae). Plants and aphids were then stored in a climatized chamber (16 hours of light/day, 25° C., 40-60% relative atmospheric humidity). After 3 and 6 days of storage, the root-systemic effect of the preparation on the aphids was determined. At a concentration of 30 ppm (based on the content of active compound), the preparations of Example Nos. 1, 2, 3, 4, 5, 6, 7, 8, 10, 11, 12, 13, 14, 15, 16, 17, 18, 20, 21, 22, 23, 24, 25, 27, 28, 37, 40, 42, 53, 54, 55, 57, 64, 66, 84, 87, 89, 90, 94, 97, 108, 110, 114, 120, 121, 122, 123, 175, 181, 182, 183, 184, 185, 191, 193, 195, 196, 202, 204, 209, 217, 218, 220, 221, 222, 223, 225, 226, 227, 228, 229, 230, 231, 232, 233, 234, 235, 237, 243, 244, 245, 249, 264, 356, 357, 359, 376, 378, 379, 380, 429, 453, 455, 458, 464, 478, 484, 500, 539, 540, 541, 542, 543, 708, 709, 712, 718 and 719 caused a mortality of 90-100% among the aphids, by root-systemic action.

### Example 3

Bush beans were transferred into brown glass bottles filled with tap water. After 5 days, adults of the white fly (Trialeurodes vaporariorum) were placed onto the beans for 48 hours, for oviposition. The adults were then removed and an aqueous solution of the formulated preparation to be examined was pipetted into the brown glass bottle. The populated plants were then stored in a climatized chamber (16 hours of light/day, 25° C., 40-60% relative atmospheric humidity). After 12 days of storage, the root-systemic effect of the preparation on the eggs was determined. At a concentration of 30 ppm (based on the content of active compound), the preparations of Example Nos. 1, 2, 3, 4, 5, 6, 7, 8, 16, 122, 196, 197, 359 and 464 caused a mortality of 90-100% among the eggs or the larvae of the aphids, by root-systemic action.

### Example 4

Rice seedlings were transferred into brown glass bottles filled with tap water. When the roots had reached a length of 5-6 cm, an aqueous solution of the formulated preparation to be examined was pipetted into the brown glass bottle. The rice was then populated with L3 larvae of the rice leaf hopper species Nephotettix cincticeps. The populated plants were then stored in a climatized chamber (16 hours of light/day, 25° C., 40-60% relative atmospheric humidity). After 4 days of storage, the root-systemic effect of the preparation on the leaf hoppers was determined. At a concentration of 30 ppm (based on the content of active compound), the preparations of Example Nos. 2, 5 and 7 caused a mortality of 90-100% among the leaf hoppers, by root-systemic action.

What is claimed is:

1. An acylsulfimide of the formula (I) and salts thereof, 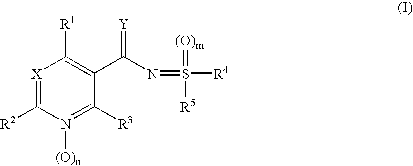
where the symbols and indices are as defined below:
X is CH or N;
Y is O or S;
n is 0 or 1;
m is 0 or 1;
R¹ is C₁-C₆-haloalkyl;
R², R³ are identical or different and are H, halogen or a branched or unbranched (C₁-C₆)-alkyl group, where one or two CH₂ groups may be replaced by —O— or —S— or —N(C₁-C₆)-alkyl, with the proviso that heteroatoms may not be adjacent to one another;
R⁴, R⁵ are identical or different and are R⁶, —C(LW)R⁷, —C(═NOR⁷)R⁷, —C(═NNR⁷₂)R⁷, —C(═W)OR⁷, —C(═W)NR⁷₂, —OC(═W)R⁷, —OC(═W)OR⁷, —NR⁷C(═W)R⁷, —N[C(═W)R⁷]₂, —NR¹C(—W)OR⁷, —C(═W)NR⁷—NR⁷₂, —C(═W)NR⁷—NR⁷[C(═W)R⁷], —NR⁷—C(═W)NR⁷₂, —NR⁷—NR⁷C(═W)R⁷, —NR⁷—N[C(═W)R⁷]₂, —N[(C═W)R⁷]—NR⁷₂, —NR⁷—NR⁷[(C═W)WR⁷], —NR⁷[(C═W)NR⁷₂], —NR⁷(C═NR⁷)R⁷, —NR⁷(C═NR⁷)NNR⁷₂, —O—NR⁷₂—O—NR⁷(C═W)R⁷, —SO₂NR⁷₂, —NR⁷SO₂R⁷, —SO₂OR⁷, —OSO₂R⁷, —OR⁷, —NR⁷₂, —SR⁷, —SiR⁷₃, —PR⁷₂, —P(═W)R⁷, —SOR⁷, —SO₂R⁷, —PW₂R⁷₂, —PW₃R⁷₂;
or
R⁴ and R⁵ together with the sulfur to which they are attached form a three- to eight-membered saturated or unsaturated ring system which is optionally mono- or polysubstituted, and which optionally contains 1 to 4 further heteroatoms, where two or more of the substituents optionally form one or more further ring systems;
W is O or S;
R⁶ are identical or different and are (C₁-C₂₀)alkyl, (C₂-C₂₀)alkenyl, (C₂-C₂₀) alkynyl, (C₃-C₈)-cycloalkyl, (C₄-C₈>cycloalkenyl, (C₈-C₁₀)-cycloalkynyl, aryl or heterocyclyl, where the radicals mentioned may optionally be mono- or polysubstituted, and
R⁷ is identical or different and is H or R⁶.

2. An acylsulfimide as claimed in claim 1, where
X is CH.

3. An acylsulfimide as claimed in claim 1, where
Y is O.

4. An acylsulfimide as claimed in claim 1, where
n is 0.

5. An acylsulfimide as claimed in claim 1, where
R¹ is (C₁-C₆)-alkyl which is mono- or polysubstituted by F and/or Cl.

6. An acylsulfimide as claimed in claim 1, where the radicals R⁴, R⁵ are substituted by one or more radicals R⁸ and where R⁸ has the following meaning:
R⁸ are identical or different and are R⁹, or two radicals R⁸ together with the atoms to which they are attached form a three- to eight-membered saturated or unsaturated ring system, optionally substituted by one or more radicals R⁹, which optionally also contains further heteroatoms;
R⁹ are identical or different and are R¹⁰, R¹¹, —C(W)R¹⁰, —C(═NOR¹⁰)R¹⁰, —C(═NNR¹⁰₂)R¹⁰, —C(═W)OR¹⁰, —C(═W)NR¹⁰₂, —OC(═W)R¹⁰, —OC(═W)OR¹⁰, —NR¹⁰C(═W)R¹⁰, —N[C(═W)R¹⁰]₂, —NR¹⁰C(═W)OR¹⁰, —C(═W)NR¹⁰—NR¹⁰₂, —C(═W)NR¹⁰—NR¹⁰ [C(═W)R¹⁰], —NR¹⁰—C(═W)NR¹⁰₂, —NR¹⁰—NR¹⁰C(═W)R¹⁰, —NR¹⁰—N[C(═W)R¹⁰]₂, —N[(C═W)R¹⁰]—NR¹⁰₂, —NR¹⁰—N[(C═W)WR¹⁰], —NR¹⁰[(C═W)NR¹⁰₂], —NR¹⁰(C═NR¹⁰)R¹⁰, —NR¹⁰(C═NR¹⁰)NR¹¹, —O—NR¹⁰₂, —O—NR¹⁰(C═W)R¹⁰, —SO₂NR¹⁰₂, —NR¹⁰SO₂R¹⁰, —SO₂OR¹⁰, —OSO₂R¹⁰, —OR¹⁰, —NR₁₂, —SR¹⁰, —SiR¹⁰ ₃, —PR¹⁰₂, —P(═W)R¹⁰ ₂, —SOR¹⁰, —SO₂R¹⁰, —PW₂R¹⁰₂, —PW₃R¹⁰₂; or two radicals R⁹ together form (═W), (═NR¹⁰), (═CR₂¹⁰), (═CHR¹⁰) or (═CH₂);
R¹⁰ are identical or different and are (C₁-C₆)-alkyl, (C₂-C₆)-alkenyl, (C₂-C₆)-alkynyl, (C₃-C₈)-cycloalkyl, (C₄-C₈)-cycloalkenyl, (C₃-C₈)-cycloalkyl-(C₁-C₄)alkyl, (C₄-C₈) cycloalkenyl-(C₁-C₄)alkyl, (C₃-C₈)-cycloalkyl-(C₁-C₄)-alkenyl, (C₄-C₈)cycloalkenyl-(C₂-C₄)-alkenyl, (Cl₁—C₆)-alkyl-(C₃-C₈)-cycloalkyl, (C₂-C₆)-alkenyl-(C₃-C₈)cycloalkyl, (C₂-C₆)-alkynyl-(C₃-C₈)-cycloalkyl, (C₁-C₆)-alkyl-(C₄-C₈)-cycloalkenyl, (C₂-C₆)-alkenyl-(C₄-C₈)-cycloalkenyl, aryl, heterocyclyl;
where the radicals mentioned are optionally-substituted by one or more radicals R¹¹; and
R¹¹ are identical or different and are halogen, cyano, nitro, hydroxyl, thio, amino, formyl, (C₁-C₆)-alkanoyl, (C₁-C₆)alkoxy, (C₃-C₆)alkenyloxy, (C₃-C₆)-alkynyloxy, (C₁-C₆)haloalkyloxy, (C₃-C₆)-haloalkenyloxy, (C₃-C₆)-haloalkynyloxy, (C₃-C₈)-cycloalkoxy, (C₄-C₈)-cycloalkenyloxy, (C₃-C₈)-halocycloalkoxy, (C₄-C₈)-halocycloalkenyloxy, (C₃-C₈-cycloalkyl-(C₁-C₄)-alkoxy, (C₄-C₈)cycloalkenyl-(C₁-C₄)-alkoxy, (C₃-C₈)-cycloalkyl-(C₂-C₄)-alkenyloxy, (C₄-C₈)-cycloalkenyl-(C₁-C₄)-alkenyloxy, (C₁-C₆)-alkyl-(C₃-C₈)cycloalkoxy, (C₂-C₆)alkenyl-(C₃-C₈)-cycloalkoxy, (C₂-C₆)alkynyl-(C₃-C₈) cycloalkoxy, (C₁-C₆)-alkyl-(C₄-C₈)-cycloalkenyloxy,
(C₂-C₆)alkenyl-(C₄-C₈)-cycloalkenyloxy, (C₁-C₄)-alkoxy-(C₁-C₆)-alkoxy, (C₁-C₄) alkoxy-(C₃-C₆)-alkenyloxy, carbamoyl, (C₁-C₆)-mono- or dialkylcarbamoyl, (C₁-C₆) mono- or dihaloalkylcarbamoyl,
(C₃-C₈)-mono- or dicycloalkylcarbamoyl, (C₁-C₆)-alkoxycarbonyl, (C₃-C₈)— cycloalkoxycarbonyl, (C₁-C₆)-alkanoyloxy, (C₃-C₈)-cycloalkanoyloxy, (C₁-C₆)— haloalkoxycarbonyl, (C₁-C₆)haloalkanoyloxy, (C₁-C₆)-alkanamido, (C₁-C₆)-haloalkanamido, (C₂-C₆)-alkanamido, (C₃-C₈)-cycloalkanamido, (C₃-C₈)-cycloalkyl-(C₁-C₄)-alkanamido, (C₁-C₆)-alkylthio, (C₃-C₆)-alkenylthio, (C₃-C₆)-alkynylthio, (C₁-C₆)-haloalkylthio, (C₃-C₆)-haloalkenylthio, (C₃-C₆)-haloalkynylthio, (C₃-C₈)-cycloalkylthio, (C₄-C₈)-cycloalkenylthio, (C₃-C₈)-halocycloalkylthio, (C₄-C₈)— halocycloalkenylthio, (C₃-C₈)-cycloalkyl-(C₁-C₄)-alkylthio, (C₄-C₈)cycloalkenyl-(C₁-C₄)-alkylthio, (C₃-C₈)-cycloalkyl-(C₃-C₄>alkenylthio, (C₄-C₈)cycloalkenyl-(C₃-C₄)-alkenylthio, (C₁-C₆)-alkyl-(C₃-C₈)-cycloalkylthio, (C₂-C₆)-alkenyl-(C₃-C₈)-cycloalkylthio, (C₂-C₆)-alkynyl-(C₃-C₈-Cycloalkylthio, (C₁-C₆)alkyl-(C₄-C₈)-cycloalkenylthio, (C₂-C₆)-alkenyl-(C₄-C₈)cycloalkenylthio, (C₁-C₆)-alkylsulfinyl, (C₃-C₆)-alkenylsulfinyl, (C₃-C₆)-alkynylsulfinyl, (C₁-C₆)-haloalkylsulfinyl, (C₃-C₆)— haloalkenylsulfinyl, (C₃-C₆)-haloalkynylsulfinyl, (C₃-C₈)-cycloalkylsulfinyl, (C₄-C₈) cycloalkenylsulfinyl, (C₃-C₈)halocycloalkylsulfinyl, (C₄-C₈)-halocycloalkenylsulfinyl, (C₃-C₈)cycloalkyl-(C₁-C₄)-alkylsulfinyl, C₄-C₈)-cycloalkenyl-(C₁-C₄)-alkylsulfinyl), (C₃-C₈)-cycloalkylsulfinyl-(C₃-C₄)-alkenylsulfinyl, (C₄-C₈)-cycloalkenyl-(C₃-C₄)alkenylsulfinyl, (C₁-C₆)-alkyl-(C₃-C₈)-cycloalkylsulfinyl, (C₂-C₆)-alkenyl-(C₃-C₈)-cycloalkylsulfinyl, (C₂-C₆)-alkynyl-(C₃-C₈)-cycloalkylsulfinyl, (C₁-C₆-alkyl-(C₄-C₈)-cycloalkenylsulfinyl, (C₂-C₆)-alkenyl-(C₄-C₈)-cycloalkenylsulfinyl, (C₁-C₆)-alkylsulfonyl, (C₃-C₆>alkenylsulfonyl, (C₃-C₆)-alkynylsulfonyl, (C₁-C₆)-haloalkylsulfonyl, (C₃-C₆)-haloalkenylsulfonyl, (C₃-C₆)— haloalkynylsulfonyl, (C₃-C₈)-cycloalkylsulfonyl, (C₄-C₈)-cycloalkenylsulfonyl, (C₃-C₈)-halocycloalkylsulfonyl, (C₄-C₈)-halocycloalkenylsulfonyl, (C₃-C₈)cycloalkyl-(C₁-C₄)-alkylsulfonyl, (C₄-C₈)-cycloalkenyl-(C₁-C₄)-alkylsulfonyl, (C₃-C₈)-cycloalkyl-(C₃-C₄)-alkenylsulfonyl, (C₄-C₈)-cycloalkenyl-(C₃-C₄)-alkenylsulfonyl, (C₁-C₆)alkyl-(C₃-C₈)-cycloalkylsulfonyl, (C₂-C₆)-alkenyl-(C₃-C₈)-cycloalkylsulfonyl, (C₂-C₆) alkenyl-(C₃-C₈)-cycloalkylsulfonyl, (C₁-C₆)alkyl-(C₄-C₈)-cycloalkenylsulfonyl, (C₂-C₆)-alkenyl-(C₄-C₈)cycloalkenylsulfonyl, (C₁-C₆)-dialkylamino, (C₁-C₆)-alkylamino, (C₃-C₆)-alkenylamino, (C₃-C₆)-alkynylamino, (C₁-C₆)-haloalkylamino, (C₃-C₆) haloalkenylamino, (C₃-C₆)-haloalkynylamino, (C₃-C₈)-cycloalkylamino, (C₄-C₈)-cycloalkenylamino, (C₃-C₈)-halocycloalkylamino, (C₄-C₈-halocycloalkenylamino, (C₃-C₈)-cycloalkyl-(C₁-C₄)-alkylamino, (C₄-C₈)-cycloalkenyl-(C₁-C₄)alkylamino, (C₃-C₈)cycloalkyl-(C₃-C₄)-alkenylamino, (C₄-C₈)-cycloalkenyl-(C₃-C₄)-alkenylamino, (C₁-C₆-alkyl-(C₃-C₈)-cycloalkylamino, (C₂-C₆)alkenyl-(C₃-C₈) cycloalkylamino, (C₂-C₆)-alkynyl-(C₃-C₈)-cycloalkylamino, (C₁-C₆)-alkyl-(C₄-C₈)-cycloalkenylamino, (C₂-C₆)-alkenyl-(C₄-C₈)-cycloalkenylamino, (C₁-C₆)-trialkylsilyl, aryl, aryloxy, arylthio, arylamino, aryl-(C₁-C₄)-alkoxy, aryl-(C₃-C₄)-alkenyloxy, aryl-(C₁-C₄)-alkylthio, aryl-(C₂-C₄)-alkenylthio, aryl-(C₁-C₄)alkylamino, aryl-(C₃-C₄)-alkenylamino, aryl-(C₁-C₆)-dialkylsilyl, diaryl-(C₁-C₆)-alkylsilyl, triarylsilyl and 5- or 6-membered heterocyclyl, the cyclic moiety of the fourteen last-mentioned radicals being optionally substituted by one or more radicals selected from the group consisting of halogen, cyano, nitro, amino, hydroxyl, thio, (C₁-C₄>alkyl, (C₁-C₄′-haloalkyl, (C₃-C₈)-cycloalkyl, (C₁-C₄)-alkoxy, (C₁-C₄)haloalkoxy, (C₁-C₄)alkylthio, (C₁-C₄)-haloalkylthio, (C₁-C₄>alkylamino, (C₁-C₄)-haloalkylamino, formyl and (C₁-C₄)alkanoyl.

7. An acylsulfimide as claimed in claim 1, where the unit SR⁴R⁵ is represented through the following structures from the group A to E: 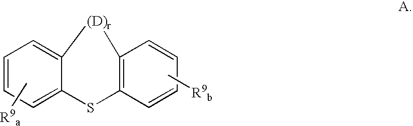
wherein the symbols and indices have the following meanings:
r is 0, 1;
D is a direct bond, (C₁-C₄)-alkylene, branched or unbranched, O, S(O)_{0,1,2}, or NR¹¹;
R⁹ is a substituent as defined in claim 6;
R¹¹ is H, (C₁-C₄)-alkyl, branched or unbranched, (C₁-C₄)-alkanoyl, (C₁-C₄)— alkoxycarbonyl, (C₁-C₄)-alkyl- or -dialkylaminocarbonyl or (C₁-C₄)-alkylsulfonyl; 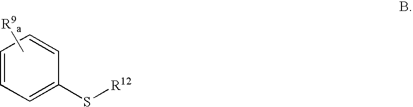
wherein the symbols and indices have the following meanings:
R¹² is (C₁-C₈)-alkyl, optionally substituted by an optionally substituted phenyl radical or (C₃-C₆)cycloalkyl radical, (C₃-C₆)-cycloalkyl, optionally substituted by or condensed with an optionally substituted phenyl radical;
R⁹ is a substituent as defined in claim 6;
a is 0, 1, 2, 3, 4, or 5, preferably 0, 1 or 2; 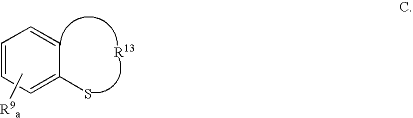
wherein the symbols and indices have the following meanings:
R⁹ is a substituent as defined in claim 6;
a is 0, 1, 2, 3 or 4, preferably 0, 1 or 2;
R¹³ is a straight chain or branched (C₂-C₈)alkanediyl group, optionally substituted by one or two or condensed with an optionally substituted phenyl radical; 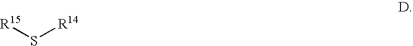
wherein the symbols and indices have the following meanings:
R¹⁴, R¹⁵ are identical or different and are in each case (C₁-C₈)-alkyl, optionally substituted by or condensed with an optionally substituted phenyl radical or (C₃-C₈)-cycloalkyl radical, (C₃-C₆Cycloalkyl, optionally substituted by or condensed with an optionally substituted phenyl radical;
and 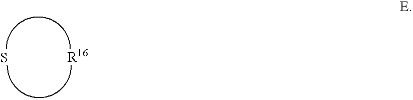
wherein the symbol has the following meaning:
R¹⁶ is a straight chain or branched (C₂-C₈)-alkanediyl group, optionally substituted by one or two or condensed with an optionally substituted phenyl radical.

8. A process for preparing a compound of formula (I) as claimed in claim 1, wherein Y is oxygen, where a carboxylic acid of the formula (V), 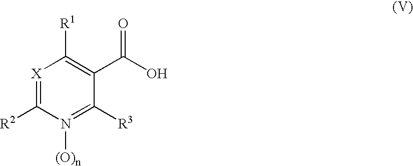
in which R¹, R², R³, X and n are as defined under formula (I) in the form of an activated derivative of this acid is reacted in the presence of a base with a compound of the formula (VI), in which R⁴, R⁵ and m are as defined under formula (I)

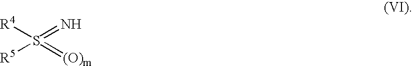

9. A composition having insecticidal, acaricidal and/or nematicidal action, which comprises at least one compound of the formula (I) as claimed in claim 1.

10. A composition having insecticidal, acaricidal and/or nematicidal action as claimed in claim 9 in a mixture with carriers and/or surfactants.

11. The composition as claimed in claim 9, which comprises a further active compound selected from the group consisting of acaricides, fungicides, herbicides, insecticides, nematicides or growth-regulating substances.

12. A veterinary medicament comprising a compound as claimed in claim 1.

13. A method for controlling harmful insects, acarids and nematodes, which comprises applying an effective amount of a compound as claimed in claim 1 to the site where the action is desired.

14. A method for controlling harmful insects, acarids and nematodes, which comprises applying an effective amount of a composition as claimed in claim 9 to the site where the action is desired.

15. A method for protecting useful plants against the undesirable action of harmful insects, acarids and nematodes, which comprises using at least one of the compounds as claimed in claim 1 for treating the seed of the useful plants.

16. A method for protecting useful plants against the undesirable action of harmful insects, acarids and nematodes, which comprises using at least one of the composition as claimed in claim 9 for treating the seed of the useful plants.

17. A process for preparing N-chloro-4-trifluoromethylnicotinamide and salts thereof of the formula (IIIa) 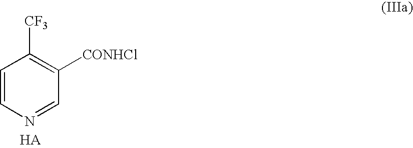
in which A is a non-oxidizable, organic or inorganic anion
by chlorination of 4-trifluoromethylnicotinamide with Cl₂ in aqueous acid and, if appropriate, subsequent anion exchange and/or, if appropriate, reaction with a base, to give N-chloro-4-trifluoromethylnicotinamide.

18. A salt of N-chloro-4-trifluoromethylnicotinamide of the formula (IIIa) 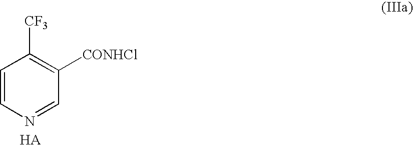
in which A is a non-oxidizable, organic or inorganic anion

19. A salt as claimed in claim 18, wherin A is F, HF₂, C₁, BF₄, PF₆, HSO₄, ½ SO₄, CH₃COO, CF₃COO, CF₃SO₃, CH₃SO₃, p-CH₃—C₆H₅SO₃ or H₂PO₄.
